(19) United States
(12) Patent Application Publication
(10) Pub. No.: US 2013/0101569 A1
(43) Pub. Date: Apr. 25, 2013

(54) HAIR GROWTH STIMULANT

(71) Applicant: Anthony R. Weston, Eagle Point, OR (US)

(72) Inventor: Anthony R. Weston, Eagle Point, OR (US)

(21) Appl. No.: 13/651,344

(22) Filed: Oct. 12, 2012

Related U.S. Application Data
(60) Provisional application No. 61/547,691, filed on Oct. 15, 2011.

(51) Int. Cl. A61K 36/889; A61K 33/06; A61K 33/24; A61K 33/34; A61K 31/202; A61K 35/60; A61K 31/519; A61K 36/16; A61K 36/258; A61K 33/18; A61K 33/32; A61K 31/455; A61K 31/197; A61K 31/525; A61K 33/04; A61K 31/51; A61K 31/07; A61K 31/714; A61K 31/4415; A61K 31/375; A61K 31/593; A61K 31/355; A61K 31/122; A61K 33/30; A61K 31/4188

(52) U.S. Cl. CPC A61K 36/889; A61K 31/4188; A61K 33/06; A61K 33/24; A61K 33/34; A61K 31/202; A61K 35/60; A61K 31/519; A61K 36/16; A61K 36/258; A61K 33/18; A61K 33/32; A61K 31/455; A61K 31/197; A61K 31/525; A61K 33/04; A61K 31/51; A61K 31/07; A61K 31/714; A61K 31/4415; A61K 31/375; A61K 31/593; A61K 31/355; A61K 31/122; A61K 33/30
USPC 424/94.1; 424/727; 424/520; 424/752; 424/728; 514/167; 514/345

(57) ABSTRACT

A composition of a mixture of vitamin and herbal supplements. Supplementation of the composition promotes hair growth and thickness by increasing the number of hairs and preventing hair loss. The concentration of the vitamin and herbal supplements in the composition is suitably about 0.01-100%. The composition may comprise a suitable carrier, solvent and/or emulgent. The composition may be, for example, an internally ingested tablet, a capsule, drops or a suspension. This formulation will enhance the hair thickness and provide elements for growing hair in humans and animals.

### FIELD OF THE INVENTION

This invention relates to the field of personal care. The said composition is for increasing hair growth and thickness with the prevention of hair loss.

### BACKGROUND OF THE INVENTION

The present invention relates to the field of scalp stimulation and in particular to the field of hair growth stimulants. Most all hair growth stimulants are topical. This invention is different from others wherein the subject is taking an oral administration of the said composition.

Topical products on the market that can actually grow hair are expensive and available only by prescription. Furthermore, cessation of use usually results in immediate hair loss. Once a user starts using this prescription product, he or she must continue for life in order to maintain hair.

Hair loss is a common occurrence between both men and women alike. Causes are due to stress, medication, weight loss, thyroid and hormonal imbalances, birth control, antibiotics, fungus, chemotherapy, and vitamin deficiency.

Hair growth is divided into the following three phases:
Anagen—hair is growing with a high level of cell proliferation occurring at the follicle.
Catagen—cell proliferation slows down its activity of hair generation.
Telogen—cell proliferation stops at the hair follicle until it sheds. If positive factors exist, the anagen phase repeats. Due to hair loss causes listed above, the normal production cycle is slowed. This may result in thinning of hair and then to baldness. (Referring FIG. 1)

The natural pattern of hair loss is gradual. As hair thins, it starts out first in the front of the scalp. Then it moves to the back and top of the head. Results with this invention have shown positive hair growth and thickness in a short period of time. A person feels more confident with thicker hair. (Referring to FIG. 2)

Hair loss is a widespread concern that is cosmetically and socially unattractive. This disorder can cause emotional distress. There is a great demand for a natural solution for hair growth and preventing hair loss. This product was designed for both men and women. Development of a simple, inexpensive composition that can grow hair represents a great improvement in the field of hair loss. This gives satisfaction to anyone with receding, thinning, or any balding hairline.

### SUMMARY OF THE INVENTION

This invention relates to a natural process of stimulating proliferation of hair follicles. Alopecia is a common problem that can cause negative psychological effects in men and women. With numerous causes ranging from stress, aging, medications, and hormone imbalances—more hair follicles remain in the third and final telogen stage. Since there is lack of positive factors to return the body to the anagen phase, the person reaches the stage of partial or complete baldness.

Hair loss is a widespread concern that is growing due to our poor diets and eating habits. With processed foods there are less vital micronutrients to sustain the body at an optimal and efficient level. This causes decreased brain and cell functions that effects the hair cycle to slow.

There are six nutrients that are considered essential to life: proteins, carbohydrates, fats, vitamins, minerals, and water. These nutrients are needed for your body to function properly, and your diet is the source of them. Vitamins and minerals are considered micronutrients because they are needed in smaller quantities than the macronutrients (protein, carbohydrates, and fat). Micronutrients do not provide calories. When your body does not absorb an adequate amount of any of the micronutrients, diseases can occur.

Vitamins are broken down into two categories: water-soluble and fat-soluble. Water-soluble vitamins dissolve in water. Leftover amounts are not stored and will leave your body through your urine. For this reason, you must consume them on a continuous basis. The water-soluble vitamins are the B vitamins and vitamin C. Fat-soluble vitamins dissolve in fat, not water. These vitamins need dietary fat in order to be absorbed in the small intestines. They are then stored in the liver and fatty tissues (adipose tissues). The fat-soluble vitamins are vitamins A, D, E, and K. [7]

Dietary minerals (also known as mineral nutrients) are the chemical elements required by living organisms, other than the four elements carbon, hydrogen, nitrogen, and oxygen present in common organic molecules. Examples of mineral elements include calcium, magnesium, potassium, sodium, zinc, and iodine. Most minerals that enter into the dietary physiology of organisms consist of simple chemical elements. Larger aggregates of minerals need to be broken down for absorption. Bacteria play an essential role in the weathering of primary minerals that results in the release of nutrients for their own nutrition and for the nutrition of others in the ecological food chain. Plants absorb dissolved minerals in soils, which are subsequently picked up by the herbivores that eat them and so on, the minerals move up the food chain.

Some sources state that sixteen chemical elements are required to support human biochemical processes by serving structural and functional roles as well as electrolytes: [1] Most of the dietary elements are of relatively low atomic weight: (Referring to FIG. 3).

Dietitians may recommend that dietary elements is best supplied by ingesting specific foods rich with the chemical element(s) of interest. The elements may be naturally present in the food (e.g., calcium in dairy milk) or added to the food (e.g., orange juice fortified with calcium). Dietary supplements can be formulated to contain several different chemical elements (as compounds), a combination of vitamins and/or other chemical compounds, or a single element (as a compound or mixture of compounds), such as calcium (as carbonate, citrate, etc.) or magnesium (as oxide, etc.), chromium (usually as picolinate). (Referring to FIGS. 4 & 5)

The dietary focus on chemical elements derives from an interest in supporting the biochemical reactions of metabolism with the required elemental components. [2] Appropriate intake levels of certain chemical elements are required to maintain optimal health. Diet can meet all the body's chemical element requirements, although supplements can be used when some requirements (e.g., calcium, found mainly in dairy products) are not adequately met by the diet, or when chronic or acute deficiencies arise from pathology, emotional or physical stress, injury, etc. [3]

The building blocks of our body during stress waste 15%-50% of protein. It's hard to rebuild our bodies during emotional or physical stress. Mass amounts of protein are now depleted from our body that is needed to generate hair, skin, and provide for the immune system. This reduction over time leaves our body in a deprived state of shock. With the lack of vital nutrients, our bodies change to a decreased state of tension. These lowered states, if not corrected, can cause the body to enter a disease state. These changes first occur at the microcellular level and later proceed to a systemic state.

Prevention of hair loss has been mostly designed without natural components. Most successful hair growth agents are synthetic and topical. Most drugs have disagreeable side effects and may not be safe or effective. This said composition is natural in content and is not hormonal or chemical. These natural materials are a higher standard that promotes hair growth and thickness. There has been no commercial success or acceptance of any natural remedies for hair loss in the form of an oral dosage. This invention gives men and women a natural choice that is safe and affordable for preventing hair loss.

### BRIEF DESCRIPTION OF THE FIGURES

The subject matter that is regarded to as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates one example of The human hair—Structure and growth cycle of the human hair. Reference [40].

FIG. 2 illustrates one example of Norwood hair model scale—The progression of male pattern baldness is generally classified on the Hamilton-Norwood scale, which ranges from stages I to VII. Reference [41].

FIG. 3 illustrates one example of Dietary elements in a periodic table—Reference [3].

FIG. 4 illustrates one example of Dietary elements—Reference [3].

FIG. 5 illustrates one example of Other Dietary elements—Reference [3].

FIG. 6 illustrates one example of Vitamin (Biotin)—Group cluster and a single soft gel/capsule of Biotin within a multivitamin.

FIG. 7 illustrates one example of Molecular formula for Biotin (C10 H16 O3 N2 S). Reference [10].

FIG. 8 illustrates one example of Vitamin (Calcium)—Group cluster and a single soft gel/capsule of Calcium.

FIG. 9 illustrates one example of Molecular formula for Calcium Carbonate (CaCO3). Reference [13].

FIG. 10 illustrates one example of Vitamin (Cholecalciferol* Vitamin D3)—Group cluster and a single soft gel/capsule of Cholecalciferol.

FIG. 11(a) illustrates one example of Molecular formula for Cholecalciferol Vitamin D3 (C27 H44 O). Reference [19].

FIG. 11(b) illustrates one example of Molecular formula for Cholecalciferol Vitamin D3 (C27 H44 O). Reference [17].

FIG. 12 illustrates one example of Molecular activation for Cholecalciferol Vitamin D3 (C27 H44 O). Reference [19].

FIG. 13 illustrates one example of Vitamin (Chromium)—Group cluster and a single soft gel/capsule of Chromium within a multivitamin.

FIG. 14 illustrates one example of Molecular formula for Chromium Picolinate (Cr(C6H4NO2)₃). Reference [42].

FIG. 15 illustrates one example of Vitamin (Copper)—Group cluster and a single soft gel/capsule of Copper within a multivitamin.

FIG. 16 illustrates one example of Molecular formula for Copper Gluconate (C12H22CuO14). Reference [43].

FIG. 17 illustrates one example of Vitamin (Docosahexaenoic acid—DHA)—Group cluster and a single soft gel/capsule of DHA within a fish oil tablet.

FIG. 18 illustrates one example of Molecular formula for Docosahexaenoic acid—DHA (C22 H32 O2). Reference [46].

FIG. 19 illustrates one example of Vitamin (Eicosapentaenoic acid—EPA)—Group cluster and a single soft gel/capsule of EPA within a fish oil tablet.

FIG. 20 illustrates one example of Molecular formula for Eicosapentaenoic acid—EPA (C20 H30 O2). Reference [50].

FIG. 21 illustrates one example of Vitamin (Omega-3 Fatty Acid)—Group cluster and a single soft gel/capsule of Omega-3 within a fish oil tablet.

FIG. 22 illustrates one example of Molecular formula for Omega-3 alpha-linolenic acid (ALA). Reference [61].

FIG. 23 illustrates one example of Herb (Ginkgo Biloba)—Group cluster and a single soft gel/capsule of Ginkgo Biloba.

FIG. 24 illustrates one example of Molecular formula for Ginkgo Biloba (C15 H18 O8) combined. Quercetin* Kaempferol* Isorhamnetin* Ginkgolides Bilobalide* Ginkgo Biloba extract 761. Reference [79] Reference [80].

FIG. 25 illustrates one example of Herb (Ginseng)—Group cluster and a single soft gel/capsule of Ginseng.

FIG. 26 illustrates one example of Molecular formula for Ginseng (C42 H72 O13). Reference [82].

FIG. 27 illustrates one example of Vitamin (Iodine)—Group cluster and a single soft gel/capsule of Iodine within a multivitamin.

FIG. 28 illustrates one example of Molecular formula for Thyroxine from Iodine (I2). Reference [88].

FIG. 29(a) illustrates one example of Iodine Benefits.

FIG. 29(b) illustrates one example of Iodine Deficiency Symptoms.

FIG. 30 illustrates one example of Vitamin (Iron)—Group cluster and a single soft gel/capsule of Iron within a multivitamin.

FIG. 31 illustrates one example of Molecular formula for Iron* within Hemoglobin (Fe). Reference [19]

FIG. 32 illustrates one example of Vitamin (Magnesium)—Group cluster and a single soft gel/capsule of Magnesium within a multivitamin.

FIG. 33 illustrates one example of Molecular formula for Magnesium Oxide (MgO). Reference [94].

FIG. 34 illustrates one example of Vitamin (Manganese)—Group cluster and a single soft gel/capsule of Manganese within a multivitamin.

FIG. 35 illustrates one example of Molecular formula for Manganese (MnSOD) Superoxide dismutase. Reference [97].

FIG. 36 illustrates one example of Vitamin (Molybdenum)—Group cluster and a single soft gel/capsule of Molybdenum within a multivitamin.

FIG. 37 illustrates one example of Molecular formula for Molybdenum Cofactor (C10 H12 MoN5 O8 PS2). Reference [100].

FIG. 38 illustrates one example of Vitamin (Pantothenic Acid* Vitamin B5)—Group cluster and a single soft gel/capsule of Pantothenic Acid* Vitamin B5 within a multivitamin.

FIG. 39 illustrates one example of Molecular formula for Pantothenic Acid* Vitamin B5 (C9 H17 NO5). Reference [104].

FIG. 40 illustrates one example of Herb (Saw Palmetto)—Group cluster and a single soft gel/capsule of Saw Palmetto.

FIG. 41 illustrates one example of Molecular formula for Saw Palmetto one of many plant sterols in extract—BETA-SITOSTEROL. Reference [121].

FIG. 42 illustrates one example of Vitamin (Selenium)—Group cluster and a single soft gel/capsule of Selenium within a multivitamin.

FIG. 43 illustrates one example of Molecular formula for Selenium Yeast (Y—Se). Illustration—None available.

FIG. 44 illustrates one example of Vitamin (Vitamin A* Retinol)—Group cluster and a single soft gel/capsule of Vitamin A* Retinol within a multivitamin.

FIG. 45 illustrates one example of Molecular formula for Vitamin A* Retinol (C20 H30 O). Reference [126].

FIG. 46 illustrates one example of Vitamin (Vitamin B1* Thiamine)—Group cluster and a single soft gel/capsule of Vitamin B1* Thiamine within a multivitamin.

FIG. 47 illustrates one example of Molecular formula for Vitamin B1* Thiamine (C12 H17 CIN4 OS). Reference [129].

FIG. 48 illustrates one example of Vitamin (Vitamin B2* Riboflavin)—Group cluster and a single soft gel/capsule of Vitamin B2* Riboflavin within a multivitamin.

FIG. 49 illustrates one example of Molecular formula for Vitamin B2* Riboflavin (C17 H20 N4 O6). Reference [133].

FIG. 50 illustrates one example of Vitamin (Vitamin B3* Niacin)—Group cluster and a single soft gel/capsule of Vitamin B3* Niacin within a multivitamin.

FIG. 51 illustrates one example of Molecular formula for Vitamin B3* Niacin (C6 NH5 O2). Reference [136].

FIG. 52 illustrates one example of Vitamin (Vitamin B6* Pyridoxine)—Group cluster and a single soft gel/capsule of Vitamin B6* Pyridoxine within a multivitamin.

FIG. 53 illustrates one example of Molecular formula for Vitamin B6* Pyridoxine (C8 H11 NO3). Reference [139].

FIG. 54 illustrates one example of Vitamin (Vitamin B9* Folic Acid)—Group cluster and a single soft gel/capsule of Vitamin B9* Folic Acid within a multivitamin.

FIG. 55 illustrates one example of Molecular formula for Vitamin B9* Folic Acid (C19 H19 N7 O6). Reference [160].

FIG. 56 illustrates one example of Vitamin (Vitamin B12* Cobalamin)—Group cluster and a single soft gel/capsule of Vitamin B12* Cobalamin within a multivitamin.

FIG. 57 illustrates one example of Molecular formula for Vitamin B12* Cobalamin (C63 H88 CoN14 O14 P). Reference [142].

FIG. 58 illustrates one example of Vitamin (Vitamin C* L-Ascorbic Acid)—Group cluster and a single soft gel/capsule of Vitamin C* L-Ascorbic Acid in a multivitamin.

FIG. 59 illustrates one example of Molecular formula for Vitamin C* L-Ascorbic Acid (C6 H8 O6). Reference [145].

FIG. 60 illustrates one example of Vitamin (Vitamin E* Tocopherol)—Group cluster and a single soft gel/capsule of Vitamin E* Tocopherol within a multivitamin.

FIG. 61 illustrates one example of Molecular formula for Vitamin E* Tocopherol (C29 H50 O2). Reference [150].

FIG. 62 illustrates one example of Vitamin (Vitamin K* Phylloquinone)—Group cluster and a single soft gel/capsule of Vitamin K* Phylloquinone within a multivitamin.

FIG. 63 illustrates one example of Molecular formula for Vitamin K* Phylloquinone (C31 H46 O2). Reference [153].

FIG. 64 illustrates one example of Vitamin (Zinc)—Group cluster and a single soft gel/capsule of Zinc Gluconate within a multivitamin.

FIG. 65 illustrates one example of Molecular formula for Zinc Gluconate (C12 H22 O14 Zn). Reference [158].

FIG. 66 illustrates one example of EMSL ANALYTICAL INC. (Nutritional Analysis)—PDF as images. PAGE 1-PAGE 5.

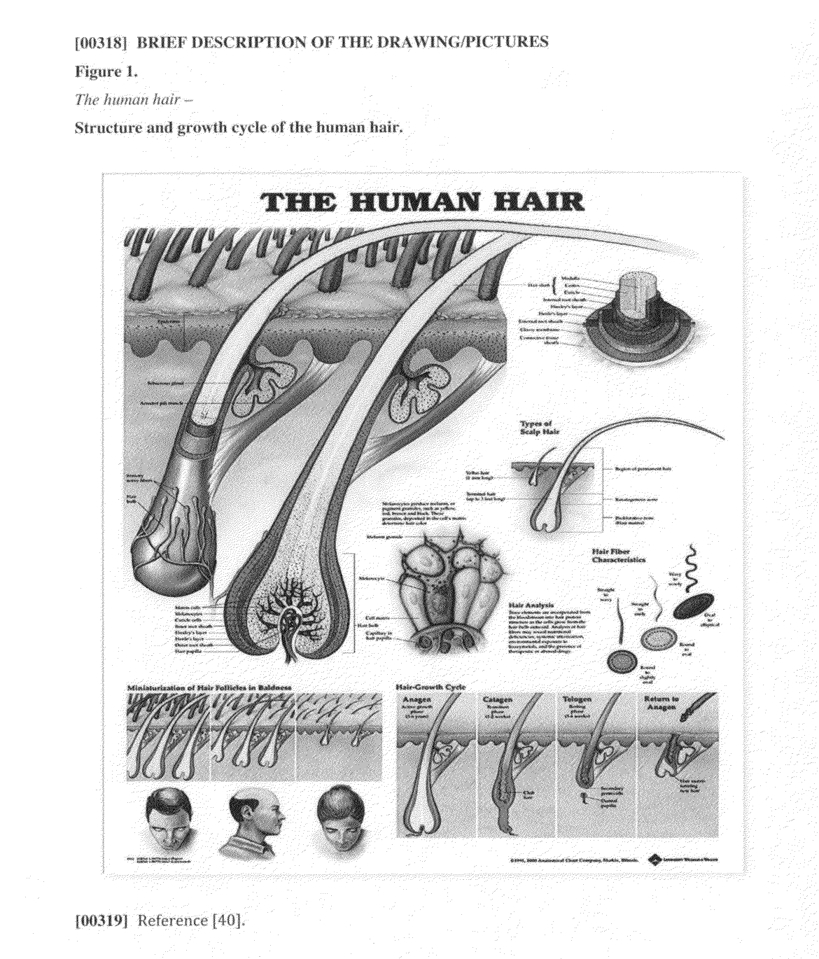

The detailed description explains the preferred embodiments of the invention, together with advantages and features, by way of example with reference to the drawings.

### DETAILED DESCRIPTION OF THE INVENTION

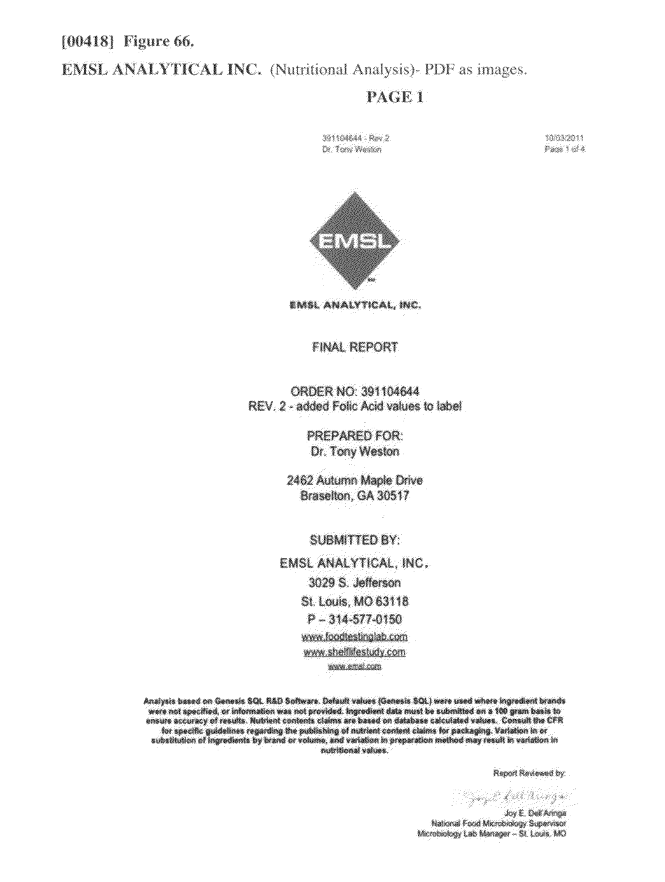

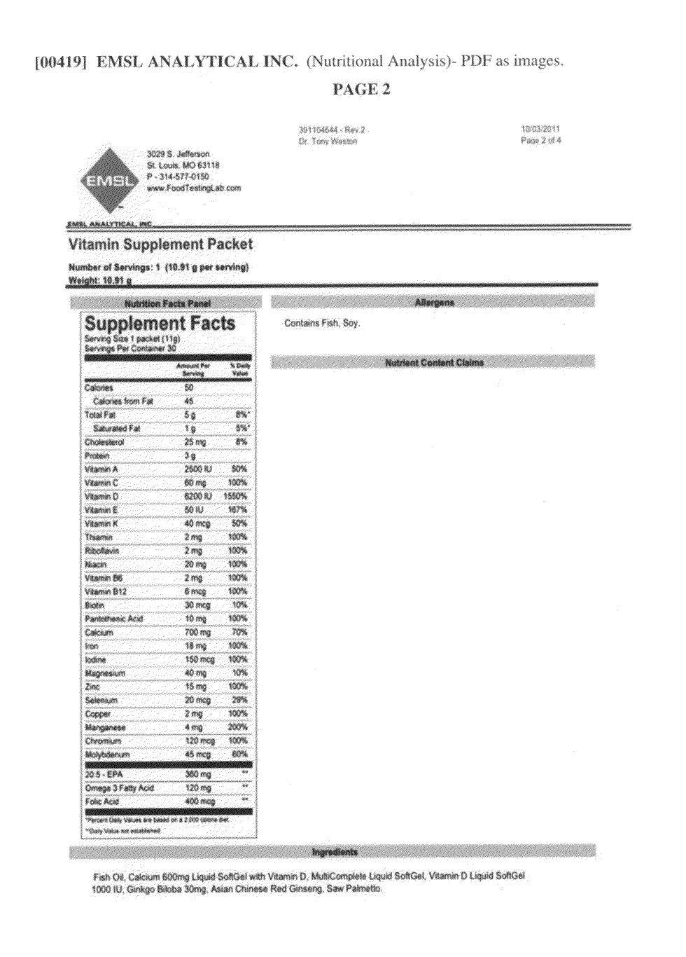

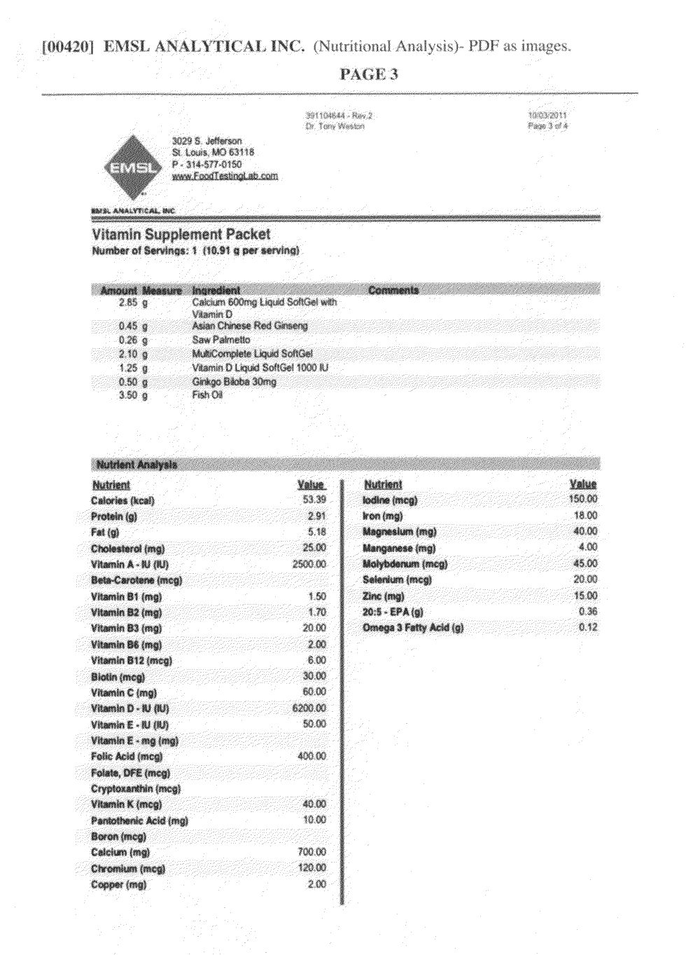

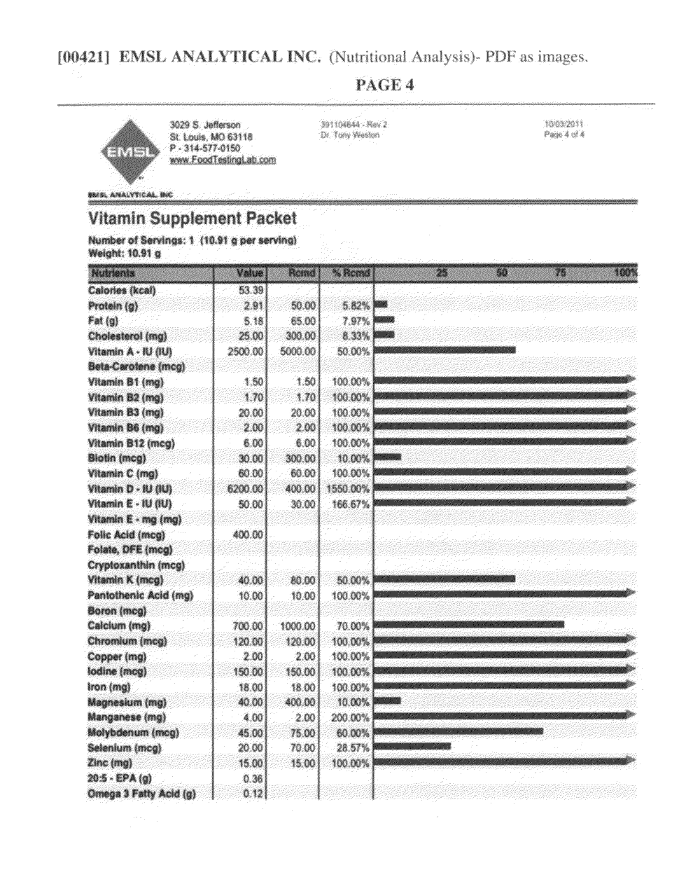

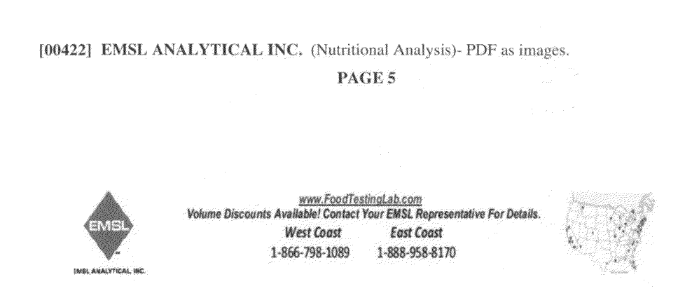

Turning now to the drawings in greater detail, it will be seen (Referring to FIG. 66), that in promoting hair growth and preventing hair loss thereof administering a composition of mixed vitamin and herbal supplements. wherein said concentration of a mixed vitamin and herbal supplements (biotin, calcium, chromium, copper, DHA-docosahexaenoic acid, EPA-Eicosapentaenoic acid, folic acid, ginkgo biloba, ginseng, iodine, iron, magnesium, manganese, molybdenum, niacin, omega-3, pantothenic acid, riboflavin, saw palmetto, selenium, thiamin, vitamin A, vitamin B12, vitamin B6, vitamin C, vitamin D—cholecalciferol, vitamin E, vitamin K, and zinc) is about 0.1%, and wherein said composition is administered in the form of an oral dosage. The composition may comprise a suitable carrier, solvent and/or emulgent.

Hair loss is growing due to our environment, poor diets, and eating habits. The following vital micronutrients combined are to sustain the body at an optimal and efficient level. As this level is achieved, normal body functions are returned. When the body and its systems are restored to a more premium state, older areas are regenerated and renewed. With this renewal, hair growth slowly returns to the scalp.

Oral dosage is in the form of an internally ingested tablet, a capsule, drops or a suspension. An oral administration of said composition is at a dosage of about 37 mg 7500 mg per day. The following shows a detailed description of each item of the said composition and its function.

Daily Amount of Biotin* Vitamin B7—dosage of about 0.3 mcg 60 mcg per day.

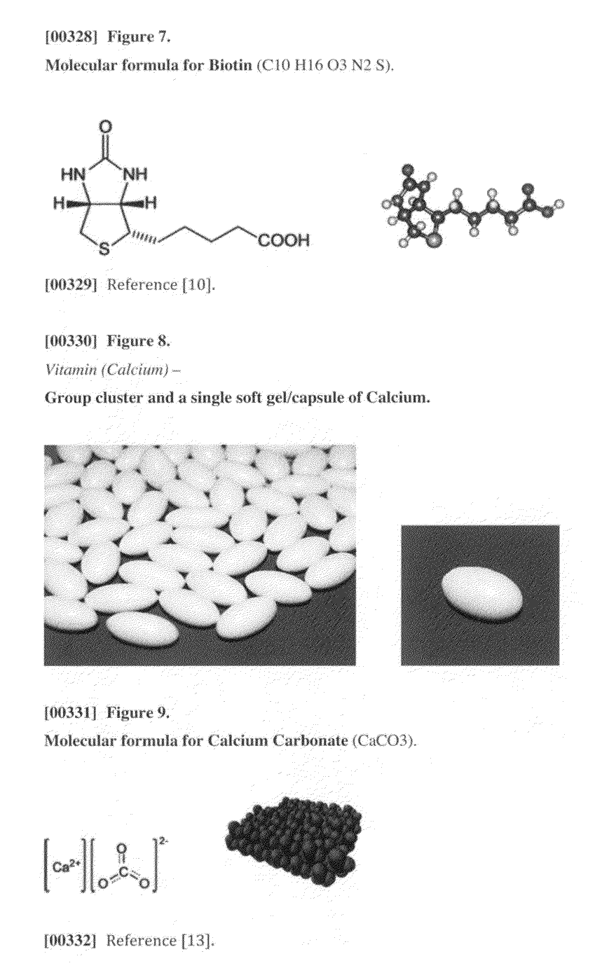

Biotin (C10 H16 O3 N2 S) (Referring to FIGS. 6-7)—[5-[(3aS,4S,6aR)-2-oxohexahydro-1H-thieno[3,4-d]imidazol-4-yl]pentanoic acid] Biotin is a water-soluble B-complex vitamin (vitamin B7).

Bateman discovered biotin in 1916.

Biotin is a heterocyclic S-containing monocarboxilic acid. Biotin D (+) is a cofactor responsible for carbon dioxide transfer in several carboxylase enzymes: Acetyl-CoA carboxylase alpha, Acetyl-CoA carboxylase beta, Methylcrotonyl-CoA carboxylase, Propionyl-CoA carboxylase, and Pyruvate carboxylase. Biotin is very important in fatty acid synthesis, branched-chain amino acid catabolism, and gluconeogenesis. [7]

Biotin is necessary for cell growth, the production of fatty acids, and the metabolism of fats and amino acids. It plays a role in the citric acid cycle, which is the process by which biochemical energy is generated during aerobic respiration. Biotin not only assists in various metabolic reactions but also helps to transfer carbon dioxide. Biotin may also be helpful in maintaining a steady blood sugar level. [5] Biotin is often recommended for strengthening hair and nails. As a consequence, it is found in many cosmetics and health products for the hair and skin.

Biotin is consumed from a wide range of food sources in the diet, however there are few rich sources. Foods with a relatively high biotin content include raw egg yolk, liver, some vegetables, and peanuts. [8] Biotin is also available from supplements. The synthetic process developed by Leo Sternbach and Moses Wolf Goldberg in the 1940s uses fumaric acid as a starting material and is identical to the natural product. [9]

Symptoms of overt biotin deficiency include: hair loss (alopecia), conjunctivitis, dermatitis in the form of a scaly red rash around the eyes, nose, mouth, and genital area. Neurological symptoms in adults are depression, lethargy, hallucination, and numbness and tingling of the extremities. [6]

The signs and symptoms of biotin deficiency include hair loss that progresses in severity to include loss of eyelashes and eyebrows in severely deficient subjects. Between 5000 mcg and 7500 mcg of biotin per day will strengthen fingernails that break, chip, or flake easily. Effects will not be noticeable for several months, depending on rate of growth. Toenails will increase in strength too, and hair will be thicker and stronger. [10]

Daily Amount of Calcium—dosage of about 7 mg 1400 mg per day.

Calcium Carbonate (CaCO3) (Referring to FIG. 8-9)—

Calcium carbonate is a chemical compound with the formula CaCO₃. It is a common substance found in rocks in all parts of the world, and is the main component of shells of marine organisms, snails, pearls, and eggshells. Calcium carbonate is the active ingredient in agricultural lime, and is usually the principal cause of hard water. It is commonly used medicinally as a calcium supplement or as an antacid.

Calcium carbonate is widely used medicinally as an inexpensive dietary calcium supplement or gastric antacid. [11] It may be used as a phosphate binder for the treatment of hyperphosphatemia (primarily in patients with chronic renal failure). It is also used in the pharmaceutical industry as an inert filler for tablets and other pharmaceuticals. [12]

Calcium carbonate is known among IBS sufferers to help reduce diarrhea. There are some individuals' reports being symptom-free since starting supplementation. The process in which calcium carbonate reduces diarrhea is by binding water in the bowel, which creates a stool that is firmer and better formed. Calcium carbonate supplements are often combined with magnesium in various proportions.

Calcium is the most abundant mineral in the human body. More than 99% of total body calcium is stored in the bones and teeth. Calcium is also found in body fluids where its function is to regulate contractions of blood vessels and muscles. The requirement for calcium is greatest from puberty to maturity, when the body grows very quickly. Milk and dairy products are good sources of calcium. [13]

Poor calcium intake can lead to bone deformation, growth retardation, bone fracture, muscle spasm, cramps, high blood pressure and osteoporosis. Calcium is a very potent sleep inducer and using it at bedtime may produce a bonus benefits for a better sleep. It aids in absorption of Vitamin B-12. It builds strong bones and teeth, prevents bone loss. It activates numbers of enzymes. And calcium regulates muscle contraction and relaxation. [14]

Daily Amount Cholecalciferol* Vitamin D—dosage of about 62 IU 12400 IU per day.

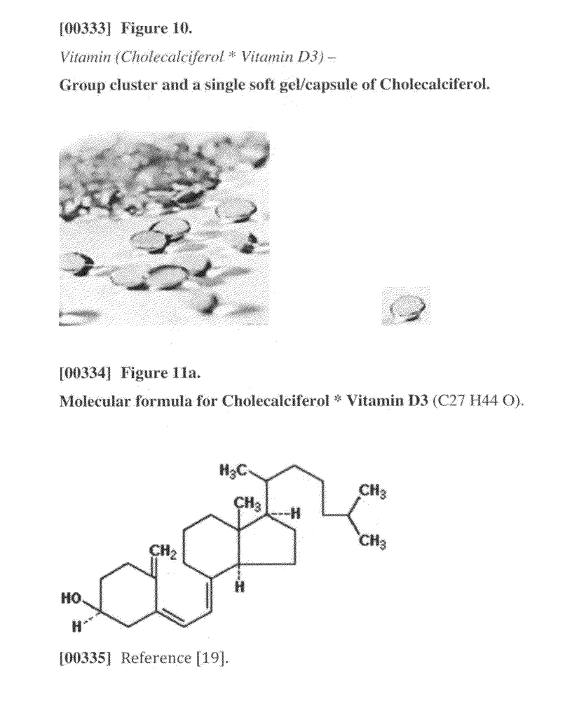

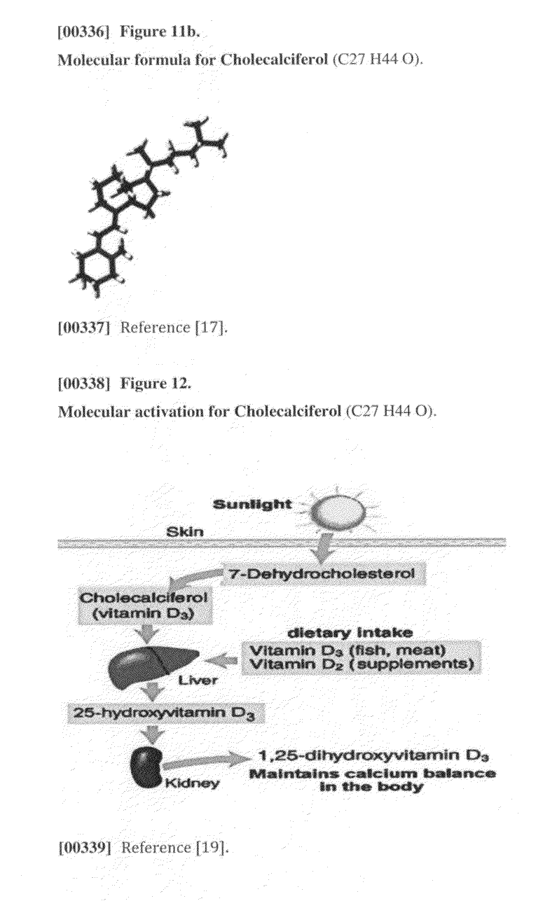

Cholecalciferol (C27 H44 O) (Referring to FIGS. 10-12)—[(3β,5Z,7E)-9,10-secocholesta-5,7,10(19)-trien-3-ol] Cholecalciferol is a form of vitamin D, also called vitamin D3 or calciol. [15][16]

It is structurally similar to steroids such as testosterone, cholesterol, and cortisol (though vitamin D₃ itself is a secosteroid). [17]

A growing body of researchers question whether the current recommended adequate levels are sufficient to meet physiological needs, particularly for individuals deprived of regular sun exposure or those at higher risk such as those with higher-melanin content in the skin (i.e. those whose ancestors are African, Latin American, or Asian), the obese, and those who live far from the equator. [18]

Vitamin D has two main forms: D2 (ergocalciferol) and D3 (cholecalciferol). Vitamin D3 is synthesized in skin by exposure to sunlight (ultraviolet radiation) and obtained in the diet chiefly in fish liver oils and egg yolks. In some developed countries, milk and other foods are fortified with vitamin D. Requirements for vitamin D increase with aging. Vitamin D is a prohormone with several active metabolites that act as hormones. Vitamin D3 is metabolized by the liver to 25(OH)D, which is then converted by the kidneys to 1,25(OH)2D (1,25-dihydroxycholecalciferol, calcitriol, or active vitamin D hormone). 25(OH)D, the major circulating form, has some metabolic activity, but 1,25(OH)2D is the most metabolically active. Inadequate exposure to sunlight may cause vitamin D deficiency. Deficiency impairs bone mineralization, causing rickets in children and osteomalacia in adults and may contribute to osteoporosis. [19]

Vitamin D is the label for a very important group of micronutrients. It acts as a steroid hormone, functioning to switch genes on and off, and helping our cells to function correctly.

Many diseases including cancer, heart disease, and diabetes have been connected with insufficient levels of vitamin D in numerous studies. In optimum amounts, it strengthens the immune system, nervous system, and bones, joints, and muscles. It can even positively affect your mood. It plays a role in reducing pain, inflammation, as well as helping to control blood sugar and blood pressure levels. [20]

Daily Amount of Chromium—dosage of about 1.2 mcg 240 mcg per day.

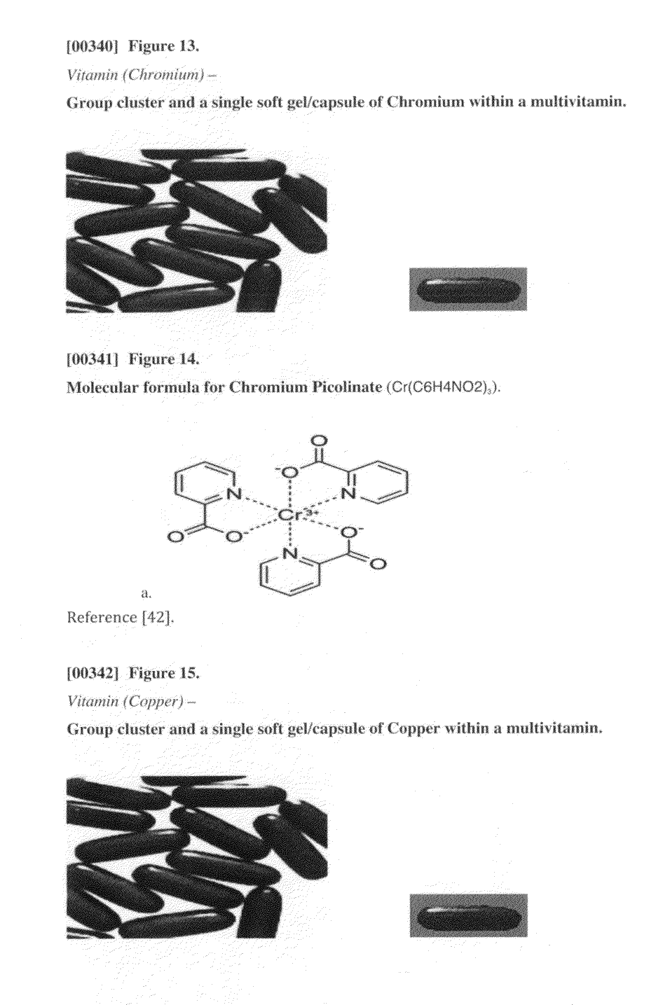

Chromium (Cr(C6H4NO2)₃) (Referring to FIG. 13-14)—Chromium(III) picolinate is the chemical compound that is sold as a nutritional supplement to prevent or treat chromium deficiency. This bright-red coordination complex is derived from chromium(III) and picolinic acid.

Chromium is a mineral that humans require in trace amounts, although its mechanisms of action in the body and the amounts needed for optimal health are not well defined. It is found primarily in two forms: trivalent (chromium III), which is biologically active and found in food. [39].

Chromium is known to enhance the action of insulin [21-23], a hormone critical to the metabolism and storage of carbohydrate, fat, and protein in the body [24]. In 1957, a compound in brewers' yeast was found to prevent an age-related decline in the ability of rats to maintain normal levels of sugar (glucose) in their blood [23]. Chromium was identified as the active ingredient in this so-called “glucose tolerance factor” in 1959 [25].

Small quantities of chromium are needed for glucose utilization by insulin in normal health, but deficiency is extremely rare and has only been observed in hospital patients on long-term defined diets. [26]

Chromium has occasionally been described as essential, [27][28] with some role in sugar metabolism in humans. Despite a market for the supplement chromium picolinate, definitive biochemical evidence for a physiological function is lacking. [29]

A more recent study focused on the effects of chromium picolinate supplementation on food intake and satiety. It indicated that chromium supplementation led to reduce cravings for fat, not carbohydrates. [30]

Chromium is widely distributed in the food supply, but most foods provide only small amounts (less than 2 micrograms [mcg] per serving). Meat and whole-grain products, as well as some fruits, vegetables, and spices are relatively good sources [31]. In contrast, foods high in simple sugars (like sucrose and fructose) are low in chromium [32].

Enhancing the mineral's absorption are vitamin C (found in fruits and vegetables and their juices) and the B vitamin niacin (found in meats, poultry, fish, and grain products) [33]. Absorbed chromium is stored in the liver, spleen, soft tissue, and bone [34].

There are reports of significant age-related decreases in the chromium concentrations of hair, sweat and blood [36], which might suggest that older people are more vulnerable to chromium depletion than younger adults [37]. One cannot be sure, however, as chromium status is difficult to determine [38]. That's because blood, urine, and hair levels do not necessarily reflect body stores. [35,37]

Daily Amount of Copper—dosage of about 0.02 mg 4 mg per day.

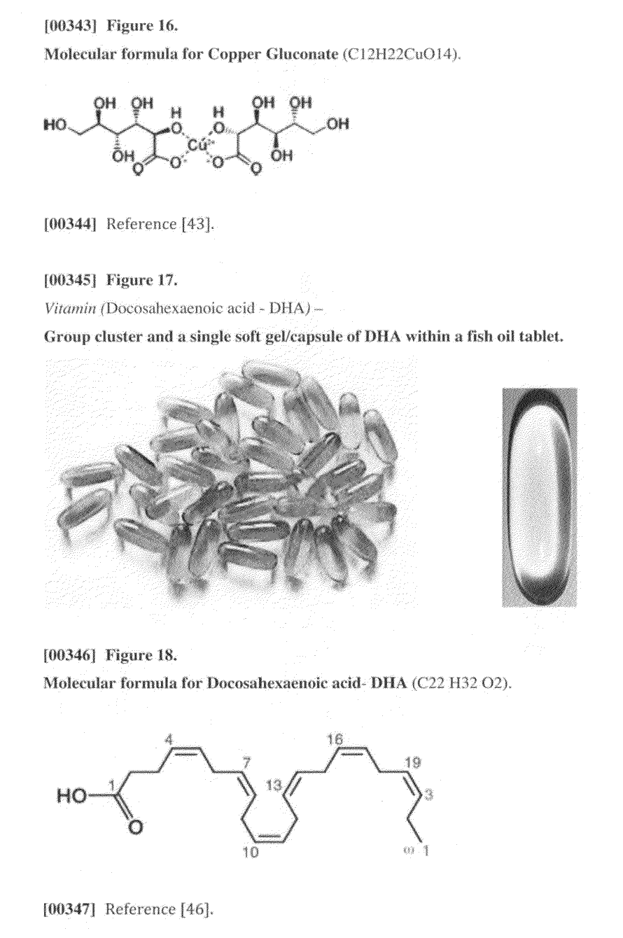

Copper Gluconate (C12H22CuO14) (Referring to FIG. 15-16)—[Copper (II) gluconate]

Copper gluconate is the copper salt of D-gluconic acid. Dietary supplement—metabolizable copper to treat copper deficiency. [43]

Copper gluconate is a form of the trace mineral copper that is most often used in copper supplements. Copper is also the third most common trace mineral in the body, yet most people, Westerners especially usually have a lack of this vital nutrient in their diets. It is vital to the body and serves many functions. [44]

Copper is essential for life. This mineral helps transports oxygen through your body, maintains healthy skin, hair color, and is used to make hormones. In fact, for a variety of biochemical processes in the body to operate normally, copper must be a part of our diet.

Copper is necessary for the growth, development, and maintenance of bone, connective tissue, brain, heart, and many other body organs. It is involved in the formation of red blood cells, the absorption and utilization of iron, and the synthesis and release of life-sustaining proteins and enzymes. These enzymes in turn produce cellular energy and regulate nerve transmission, blood clotting, and oxygen transport. Copper stimulates the immune system to fight infections, repair injured tissues, and promote healing. Copper also helps to neutralize “free-radicals”. [45]

Copper is also needed for certain critical enzymes to function in the body. These enzymes are involved with energy metabolism, the way that the body gives you the energy to function. Too little copper in the body can actually directly lead to disease. Copper is involved in the functioning of the nervous system and in maintaining the balance of other useful metals in the body such as zinc and molybdenum. It is a necessary precursor to a powerful antioxidant within the system superoxide dismutase. It therefore has antioxidant capabilities. [45]

Copper helps alleviate rheumatoid arthritis. It is an anti-inflammatory with actions that help reduce arthritis symptoms. Copper also helps promote bone health and prevent osteoporosis. It helps prevent heart disease by putting off heart rhythm disorders, and high blood pressure. [44]

Some believe copper supplements help prevent hair from graying as one of the reasons hair turns gray is due to a copper deficiency.

Due to its antioxidant activity, copper may help prevent cancer and other ailments. Those who especially need copper gluconate include people who have an eating disorder, malnourished children, alcoholics, and those who suffer constant diarrhea. Some other benefits include helping prevent prostate disorders, and hypoglycemia. Some food sources of copper include wheat, peaches, honey, potatoes, legumes, and nuts. [44]

Daily Amount of Docosahexaenoic acid* DHA in Fish Oil—dosage of about 2.4 mg 480 mg per day.

Docosahexaenoic acid (C22 H32 O2) (Referring to FIGS. 17-18)—[4Z,7Z,10Z,13Z,16Z,19Z)-docosa-4,7,10,13,16,19-hexaenoic acid; Doconexent]

Dietary DHA may reduce the risk of heart disease by reducing the level of blood triglycerides in humans. Low levels of DHA have been associated with Alzheimer's disease. [46]

Fish oil is widely sold in gelatin capsules containing a mixture of omega-3 fatty acids including EPA and smaller quantities of DHA. One study found fish oil higher in DHA than EPA lowered inflammatory cytokines, such as IL-6 and IL-1β, associated with neurodegenerative and autoimmune diseases. They note the brain normally contains DHA, but not EPA, though both DHA and EPA plasma concentrations increased significantly for participants. [47]

DHA deficiency likely plays a role in decline of mental function in healthy adults, which is indicated in a study from 2010 conducted at 19 U.S. clinical sites on 485 subjects aged 55 and older who met criteria for age-associated memory impairment. The study found algal DHA taken for six months decreased heart rate and improved memory and learning in healthy, older adults with mild memory complaints. These findings indicate the importance of early DHA intervention and provided a statistically significant benefit to cognitive function in individuals over 50 years of age. [48]

DHA is the most abundant omega-3 fatty acid in the brain and retina. DHA comprises 40% of the polyunsaturated fatty acids (PUFAs) in the brain and 60% of the PUFAs in the retina. Fifty percent of the weight of a neuron's plasma membrane is composed of DHA. [49]

Some core fish oil health benefits include: reducing the risk of heart attacks, strokes and helps correct dangerously abnormal heart rhythms. Also it improves heart health by lowering cholesterol and triglyceride levels. Fish oil raises HDL—the good cholesterol, lowers the risk of blood clots, and helps with inflammation.

It significantly reduces overall blood pressure levels and improves autoimmune diseases, and arthritis. It alleviates autoimmune diseases while helping eczema, migraines and even colitis. It improves mental health issues such as depression. It aids in eye and joint health, with the prevention of toxemia, and with brain function and health.

Furthermore, there is much evidence, as alluded, from several clinical studies that strongly suggest amounts of DHA and EPA in the form of fish or fish oil supplements lowers triglycerides and significantly slows the buildup of atherosclerotic plaques, which is essentially hardening of the arteries.

Daily Amount of Eicosapentaenoic acid* EPA in Fish Oil—dosage of about 3.6 mg 720 mg per day.

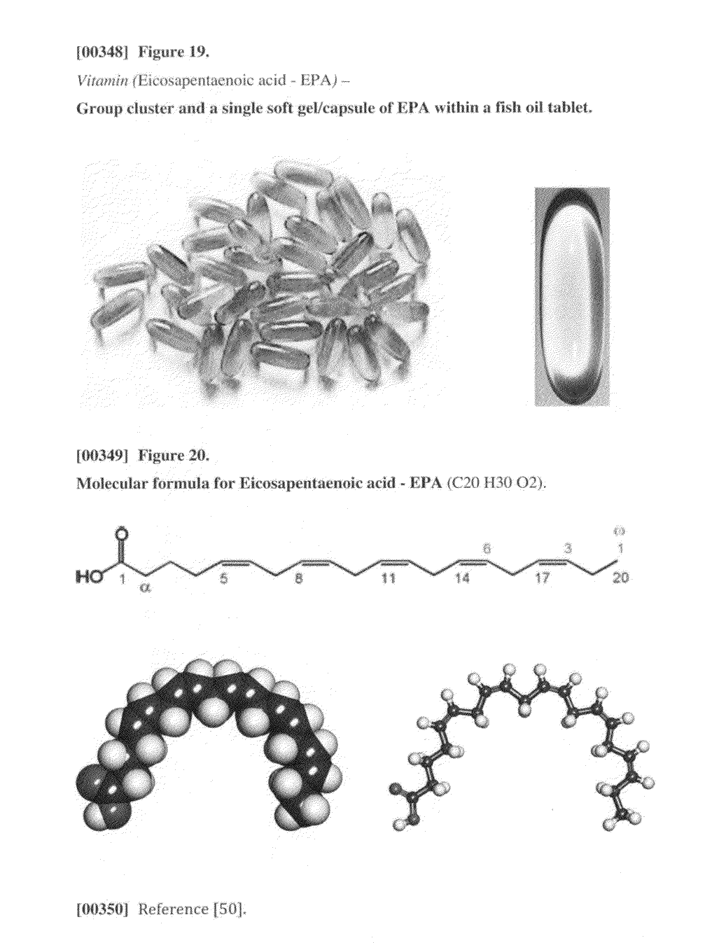

Eicosapentaenoic acid (C20 H30 O2) (Referring to FIGS. 19-20)—[(5Z,8Z,11Z,14Z,17Z)-eicosa-5,8,11,14,17-pentenoic acid]

Eicosapentaenoic acid (EPA) is an omega-3 fatty acid. EPA is a polyunsaturated fatty acid (PUFA) that acts as a precursor for prostaglandin-3 (which inhibits platelet aggregation), thromboxane-3, and leukotriene-5 groups (all eicosanoids). [50]

It is obtained in the human diet by eating oily fish or fish oil—e.g., cod liver, herring, mackerel, salmon, and sardine. It is also found in human breast milk. However, fish do not naturally produce EPA, but obtain it from the algae they consume. [51]

The US National Institute of Health's Medline Plus lists medical conditions for which EPA (alone or in concert with other ω-3 sources) is known or thought to be an effective treatment. [52] Most of these involve its ability to lower inflammation.

Among omega-3 fatty acids, it is thought that EPA in particular may possess some beneficial potential in mental conditions, such as schizophrenia. [53] [54]

EPA is required for the production of a special group of substances in the body called prostaglandins, which control blood clotting and other arterial functions. EPA also provides a natural approach to lower blood cholesterol and triglycerides.

These lipid-lowering effects, along with some benefits in reducing platelet aggregation and clotting potential, make the use of EPA/DHA very important in the treatment or prevention of cardiovascular disease or in anyone with high blood fats or low HDL. The decreased blood viscosity and lower fat levels help reduce the risk of heart attacks. The mild anti-inflammatory effects, possibly a result of increased PGE1 and PGE3 prostaglandins, may also be helpful and has suggested the possible use of EPA/DHA in arthritis and other inflammatory conditions. In rheumatoid arthritis, for example, EPA/DHA supplementation has been shown to reduce joint stiffness and soreness and to improve flexibility. [55]

People with certain circulatory problems, such as varicose veins, may benefit from such supplements containing EPA and DHA, which stimulate blood circulation, increase the breakdown of fibrin, a compound involved in clot and scar formation, and, in addition, have been shown to reduce blood pressure. [56] [57]

Several studies report possible anti-cancer effects of n-3 fatty acids (in particular, breast, colon, and prostate cancer). [58] [59] [60]

Daily Amount of Omega-3 Fatty Acid* ALA in Fish Oil—dosage of about 1.2 mg 240 mg per day.

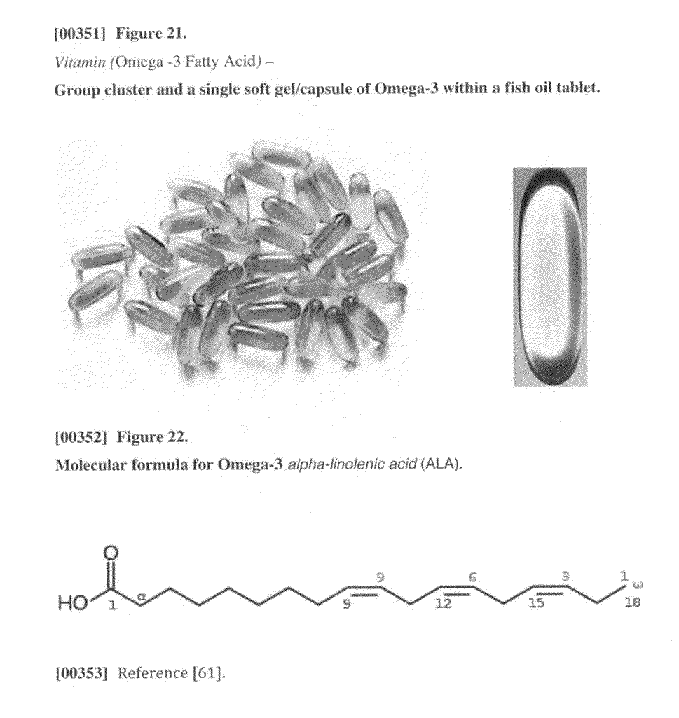

Alpha-linolenic acid (ALA) (Referring to FIG. 21-22)—[all-cis-9,12,15-octadecatrienoic acid]

N-3 fatty acids (popularly referred to as ω-3 fatty acids or omega-3 fatty acids) are essential unsaturated fatty acids with a double bond (C═C) starting after the third carbon atom from the end of the carbon chain. [61]

Fish oil is oil derived from the tissues of oily fish. Fish oils contain the omega-3 fatty acids eicosapentaenoic acid (EPA), and docosahexaenoic acid (DHA), precursors of eicosanoids that are known to reduce inflammation throughout the body [62] [63] and are thought to have many health benefits.

Essential fatty acids are molecules that cannot be synthesized by the human body but are vital for normal metabolism. One of the two families of these essential fatty acids is the omega-3 fatty acid.

Nutritionally important n-3 fatty acids include α-linolenic acid (ALA), eicosapentaenoic acid (EPA), and docosahexaenoic acid (DHA), all of which are polyunsaturated.

Common sources of n-3 fatty acids include fish oils and some plant oils such as flaxseed oil and algal oil.

Mammals cannot synthesize n-3 fatty acids, but have a limited ability to form the “long-chain” n-3 fatty acids EPA (20-carbon atoms) and DHA (22-carbon atoms) from the “short-chain” eighteen-carbon n-3 fatty acid ALA. [61]

The health benefits of the long-chain omega-3 fatty acids—DHA and EPA omega-3—are the best known. These benefits were discovered in the 1970s by researchers studying the Greenland Inuit Tribe. The Greenland Inuit people consumed large amounts of fat from seafood, but displayed virtually no cardiovascular disease. The high level of omega-3 fatty acids consumed by the Inuit reduced the triglycerides, heart rate, blood pressure, and atherosclerosis. [64]

Several studies report possible anti-cancer effects of n-3 fatty acids (in particular, breast, colon, and prostate cancer. [65] [66] [67] Omega-3 fatty acids reduced prostate tumor growth, slowed histopathological progression, and increased survival in mice.

The omega-3 fatty acids in fish oil have been studied for treating clinical depression, [68] [69] anxiety, [70] [71] and enhancing the benefits from depression medications. [72] Countries with the highest intake of fish in their diets are correlated with the lowest rates of depression among citizens. [73]

Daily Amount of Ginkgo Biloba—dosage of about 0.3 mg 60 mg per day.

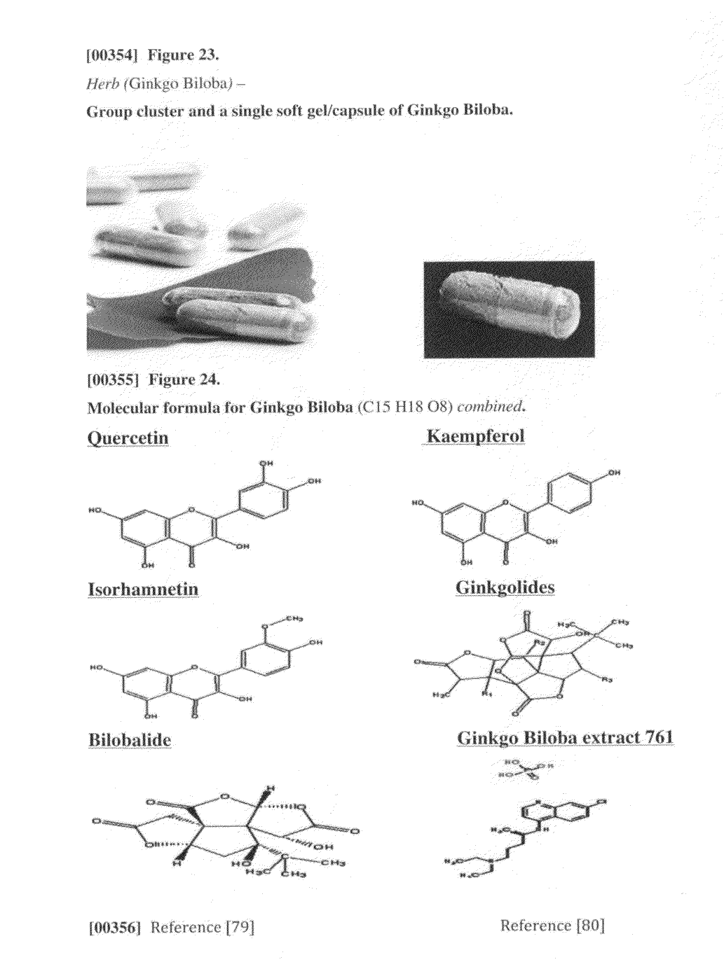

Ginkgo Biloba (C15 H18 O8) (Referring to FIG. 23-24)—

[4H,5aH,9H-Furo(2,3-b)furo(3′,2′:2,3)cyclopenta(1,2-c)furan-2,4,7(3H,8H)-trione,9-(1,1-dimethylethyl)-10,10a-dihydro-8,9-dihydroxy-, (5aR-(3aS*,5aα,8β,8aS*,9α,10aα))-(9CI)]

Ginkgo Biloba comes from the ginkgo tree, one of the oldest trees in the world, according to the National Center for Complementary and Alternative Medicine. This herb has been used to treat various conditions including asthma, fatigue, tinnitus and memory. The leaves of the ginkgo plant are used as an extract, tablet, and capsule or in a tea. Ginkgo may offer your body and mind several benefits and help increase your health. [74]

Ginkgo may help relieve or treat problems of the eye, such as macular degeneration. This condition affects the retina, the back part of your eye. Macular degeneration is progressive and commonly affects adults who are older. According to the University of Maryland Medical Center, research indicated ginkgo can help maintain healthy vision in people with macular degeneration. [74]

Extracts of Ginkgo leaves contain flavonoid glycosides and terpenoids (ginkgolides, bilobalides) and have been used pharmaceutically. Recently, careful clinical trials have shown Ginkgo to be effective in treating dementia [75] but not preventing the onset of Alzheimer's disease in normal people. [76] [77]

Ginkgo is believed to have inotropic properties, and is used as memory [78] and concentration enhancer, and anti-vertigo agent.

It is said to be effective in improving the blood flow to the hands and the feet as well as stimulating the brain and reducing short-term memory loss. It increases blood flow to the brain, the uptake of glucose by brain cells, and has been said to improve the transmission of nerve signals. [79]

In studies, Ginkgo Biloba has been reported as demonstrating anti-oxidant abilities with improvements of the platelet and nerve cell functions and blood flow to the nervous system and brain. It has also been reported as reducing blood viscosity. It's ability to increase vascular dilation, may help reduce retinal damage due to macular degradation and may reverse deafness caused by reduced blood flow. [79]

Germany, France, America have run literally hundreds of studies on the leaf extract. The extract of Ginkgo Biloba has been studied for its effectiveness in the treatment of Acrocyanosis, Alzheimer's disease, Cerebral atherosclerosis, Cerebral insufficiencies, Cochlear deafness, Dementia, Depression, Menopause, Peripheral and cerebral circulatory stimulation, Peripheral vascular disease, Raynaud's syndrome, Retinopathy, Senility, Short-term memory loss, Tinnitus, Vascular Diseases, and Vertigo. [79]

Daily Amount of Ginseng—dosage of about 2.5 mg 500 mg per day.

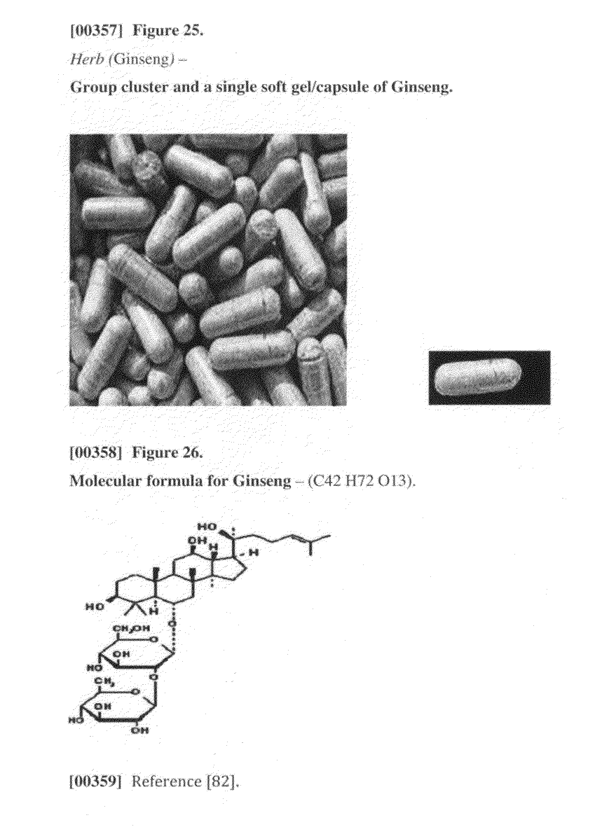

Ginseng (C42 H72 O13) (Referring to FIG. 25-26)—

Ginseng is one of nature's greatest gifts. It is used to increase physical and mental endurance, boost energy, normalize body functions, reduce cholesterol, and prevent cancer. Traditionally, ginseng has been used to enhance sexual desire, by promoting sex hormone production. Ginseng supports the natural balance of your body to combat fatigue and strengthens and protects your nervous system. Recent research shows that regular use of Ginseng is helpful in the treatment of memory loss (Alzheimer's), balance of blood sugar levels (Diabetes), slowing down the aging process, helping the immune system, etc. [81]

Ginseng may be effective for treating colds, coughs, rheumatism, neuralgia, gout, diabetes, anemia insomnia, stress, headache, backache and double vision. Ginseng also counteracts the effects of physical and emotional stress, enhances memory, counteracts fatigue without caffeine, and improves stamina. Medically, ginseng stimulates the immune system, by spurring the production of the body's own virus fighting chemicals, helps reduce cholesterol levels in the blood, has anti-clotting effects, reducing risk of arterial blood clots, helps control diabetes by reducing blood sugar levels, is known as an antioxidant, preventing the cumulative cell damage in cancer, protects the liver from the effects of drug, alcohol and toxins, minimizes cell damage from radiation, and increases intestinal absorption of nutrients. [81]

Found exclusively in the ginseng, ginsenosides are among the most sought-after steroidal saponins in nature. Ginsenosides are thought to improve resistance to stress and enhance homeostasis. They are as well purported to be of a great effect on workers, fostering their mental and physical capabilities. Recently, studies reported a close link between the uses of ginsenosides and man's fertility. [82]

Ginkgo Biloba is especially good when combined with Asian Ginseng.

Ginseng has been used to both stimulate and relax the nervous system. It increases capillary circulation in the brain and decreases the effects of stress. Though there are many kinds of ginsengs in the world but they cannot rival Asian Ginseng in ingredients and medicinal effects. It contains as many as 29 different ginsenosides while the others contains 8-9. [83]

Asian Ginseng contains anti-ageing substances such as anti-oxidants and insulin-like substances that are not found in any other type of ginseng.

Daily Amount of Iodine—dosage of about 1.5 mcg 300 mcg per day.

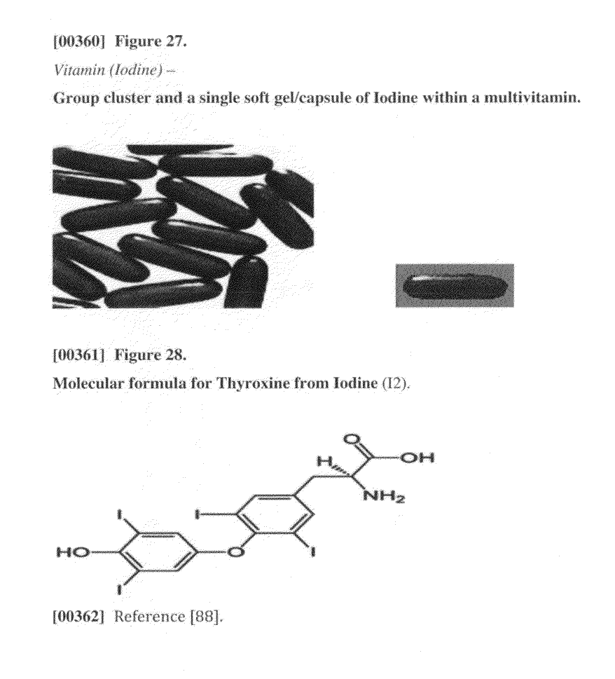

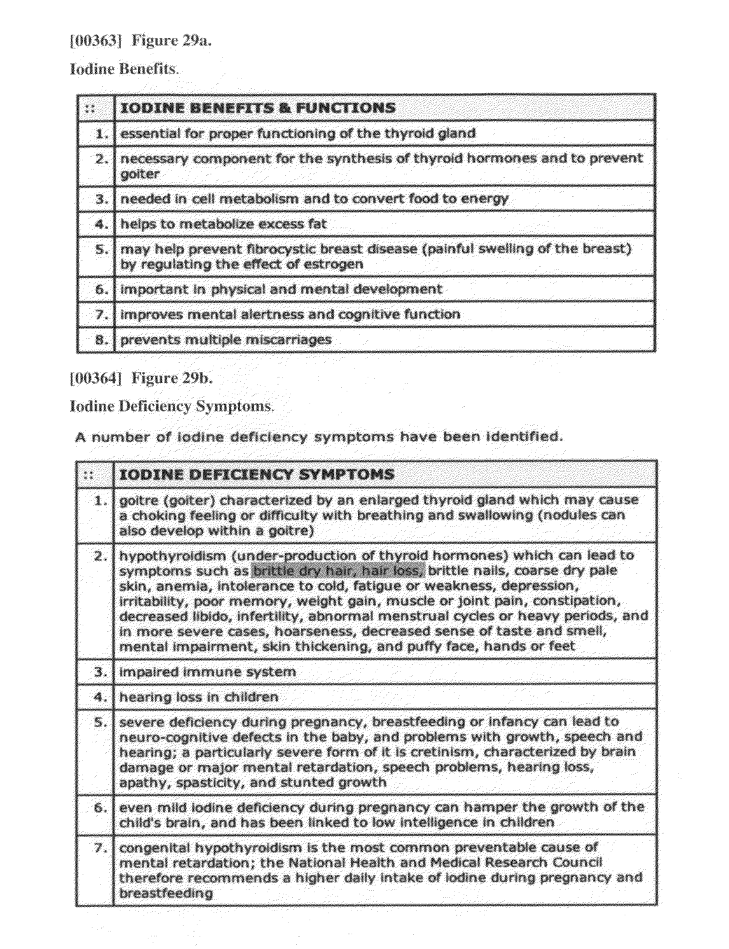

Iodine (I2) (Referring to FIG. 27-29)—Iodine is an essential trace element for life, the heaviest element needed by living organisms, and the second-heaviest to be used by any form of life.

Iodine's main role in animal biology is as a constituent of the thyroid hormones thyroxin (T4) and triiodothyronine (T3). These are made from addition condensation products of the amino acid tyrosine, and are stored prior to release in an iodine-containing protein called thyroglobulin. T4 and T3 contain four and three atoms of iodine per molecule, respectively. The thyroid gland actively absorbs iodide from the blood to make and release these hormones into the blood, actions that are regulated by a second hormone TSH from the pituitary. Thyroid hormones are phylogenetically very old molecules that are synthesized by most multicellular organisms. [84]

Thyroid hormones play a basic role in biology, acting on gene transcription to regulate the basal metabolic rate. The total deficiency of thyroid hormones can reduce basal metabolic rate up to 50%, while in excessive production of thyroid hormones the basal metabolic rate can be increased by 100%. T4 acts largely as a precursor to T3, which is the biologically active hormone.

Iodine is required not only for the synthesis of thyroid hormones, thyroxine and triiodothyronine and to prevent goiter, but also, probably as an antioxidant, for extra thyroidal organs as mammary and salivary glands and for gastric mucosa and immune system (thymus). [3]

Iodine has a nutritional relationship with selenium. A family of selenium-dependent enzymes called deiodinases converts T4 to T3 (the active hormone) by removing an iodine atom from the outer tyrosine ring.

The thyroid gland needs no more than 70 micrograms/day to synthesize the requisite daily amounts of T4 and T3. The higher recommended daily allowance levels of iodine seem necessary for optimal function of a number of body systems, including lactating breast, gastric mucosa, salivary glands, oral mucosa, thymus, epidermis, choroid plexus, etc. [85][86][87]

Iodine benefits range from proper cell metabolism to convert food to energy, metabolizing excess fat, important physical and mental development, and improves mental alertness and cognitive function. Deficiency symptoms are goiter, hypothyroidism, impaired immune system, and hearing loss in children. [88]

Hypothyroidism (under-production of thyroid hormones) which can lead to symptoms such as brittle dry hair, hair loss, brittle nails, coarse dry pale skin, anemia, depression, irritability, poor memory, weight gain, muscle or joint pain, constipation, decreased libido, infertility, abnormal menstrual cycles or heavy periods, and in more severe cases, hoarseness, decreased sense of taste and smell, mental impairment, skin thickening, and puffy face, and hands or feet. [88]

Daily Amount of Iron* ferrous sulfate—dosage of about 0.18 mg 36 mg per day.

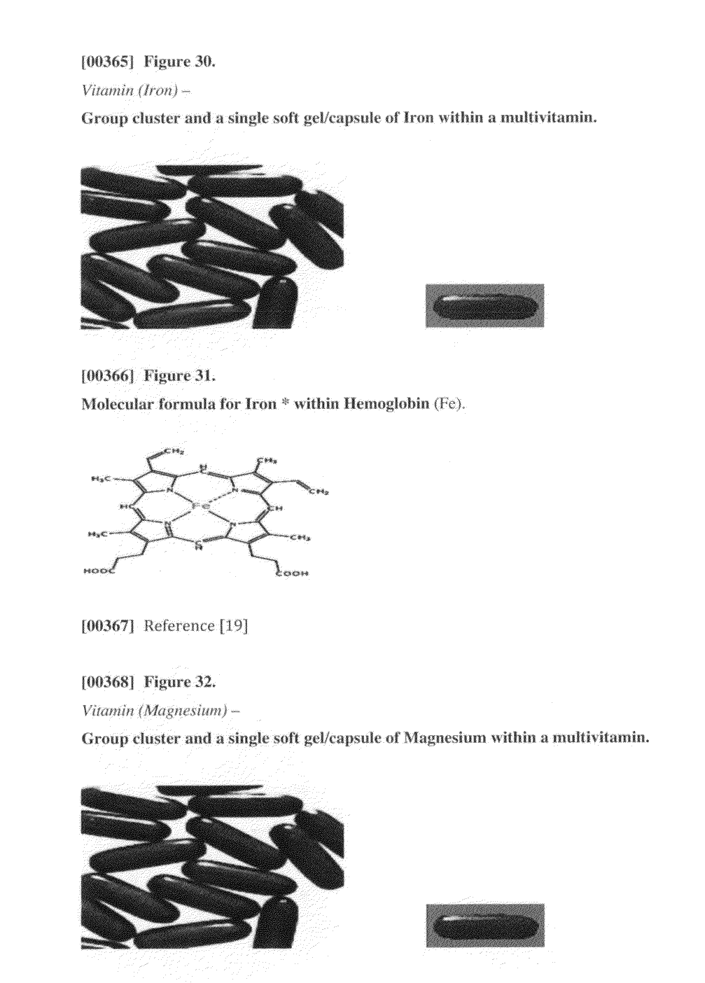

Iron II sulfate (Ferrous Sulfate) (FeSO4) (Referring to FIG. 30-31)—

Iron(II) sulphate or ferrous sulfate is the chemical compound with the formula FeSO₄. Together with other iron compounds, ferrous sulfate is used to fortify foods and to treat iron-deficiency anemia. [89]

Iron (Fe) is a component of hemoglobin, myoglobin, and many enzymes in the body. Heme iron, contained mainly in animal products, is absorbed much better than nonheme iron, which accounts for over 85% of iron in the average diet. However, absorption of nonheme iron is increased when it is consumed with animal protein and vitamin C. Iron plays an integral role in the transportation of oxygen through the bloodstream. Iron can also affect brain function as it aids in the synthesis of neurotransmitters. [19]

Iron deficiency, which can cause anemia, is the most common nutritional deficiency in the world. It may result from inadequate iron intake or from malabsorption. Meat products are the best sources of dietary iron. Iron deficiency may be caused by an improper vegan or ovo-lacto vegetarian diets. Chronic bleeding may also cause iron deficiency. [19]

There are many symptoms of anemia. Common ones are fatigue, slow cognitive functioning, depression, a swollen tongue, weakness, cold hands and feet, and brittle nails. [90]

Other symptoms and signs of iron deficiency anemia include: irritability, angina, constipation, sleepiness, tinnitus, mouth ulcers, palpitations, hair loss, fainting or feeling faint, depression, breathlessness on exertion, twitching muscles, tingling, numbness, or burning sensations, missed menstrual cycle, glossitis (inflammation or infection of the tongue), angular cheilitis (inflammatory lesions at the mouth's corners), koilonychia (spoon-shaped nails) or nails that are weak or brittle, poor appetite, and pruritus (itchiness). [91]

A review of 40 years of research shows that iron deficiency has a much closer link to hair loss than most doctors realize. It may be the key to restoring hair growth, Cleveland Clinic dermatologists find. “We believe that treatment for hair loss is enhanced when iron deficiency, with or without anemia, is treated,” Leonid Benjamin Trost, MD; Wilma Fowler Bergfeld, MD; and Ellen Calogeras, RD, MPH, May issue of the Journal of the American Academy of Dermatology. [92]

“In our clinic's experience, it is clear that if you replenish hair-loss patients' iron stores with iron supplements, they are more likely to regrow hair, or at least stop hair shedding,” Cotsarelis tells WebMD. “And they don't have to be anemic. That is the biggest mistake doctors make.” [92]

Dietary sources include red meat, leafy green vegetables, fish (tuna, salmon), eggs, dried fruits, beans, whole grains, and enriched grains. [3]

Daily Amount of Magnesium—dosage of about 0.4 mg 80 mg per day.

Magnesium Oxide (MgO) (Referring to FIG. 32-33)—[Magnesium oxide]

Magnesium is one of the most in-demand minerals for the human body and which is responsible in a variety of distinctive in keeping our body healthy, balanced and vibrant.

Magnesium is a co-factor in numerous chemical reactions. Co-factor means it's needed in the “assistance of” or is a catalyst for other functions. Magnesium is needed to make hundreds of enzymes work and assists with thousands of others. It's part of your life force. [93]

Magnesium produces and transports energy and is involved with hundreds of enzymatic reactions in the body. As a result, magnesium deficiency can affect every aspect of your physiology. Because magnesium is involved in activating the most important enzyme reaction in the body, ATP (adenosine triphosphate), without it there is no energy, no movement, no life. [93]

It synthesizes proteins. Magnesium is used in conjunction with and synthesizes many other vitamins and minerals. It transmits nerve signals. Magnesium allows a small amount of calcium to enter a nerve cell. Our thoughts, via brain neurons, are dependent on it. [93]

Magnesium is also important for the contraction and relaxation of muscles. Too much calcium and the not enough magnesium inside a cell and you can get muscle twitches, spasms, even convulsions. [93]

Magnesium is involved in the synthesis of protein, and it assists in the functioning of some enzymes. [19]

Magnesium deficiency is surprisingly long and includes: anxiety, depression, muscle weakness, fatigue, eye twitches, PMS, insomnia, anorexia, apathy, apprehension, poor memory, confusion, anger, nervousness, and rapid pulse. [93]

As one of the most inexpensive and accessible supplements on the market, magnesium is responsible for regulating 325 enzymes in the body. [93]

Dietary sources include nuts, soybeans, cocoa mass, cereals, and dark green leafy vegetables, which are rich in chlorophyll. [3] [19]

Daily Amount of Manganese—dosage of about 0.04 mg 8 mg per day.

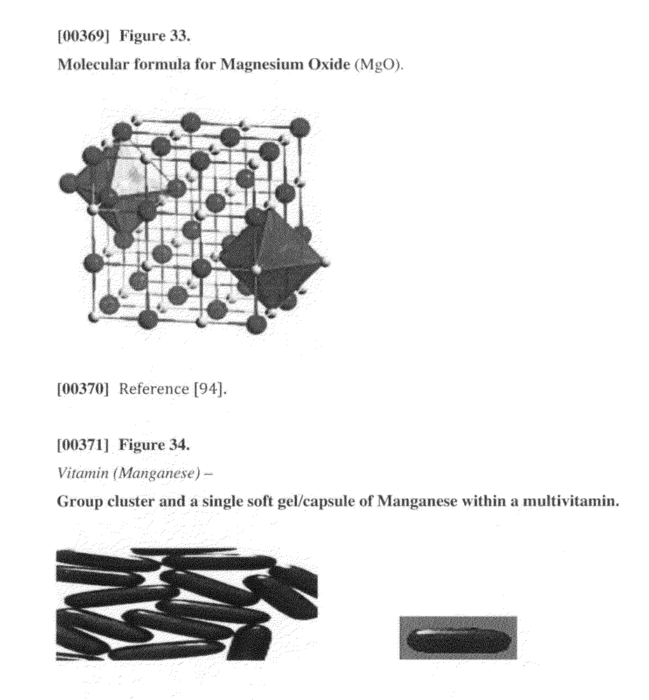

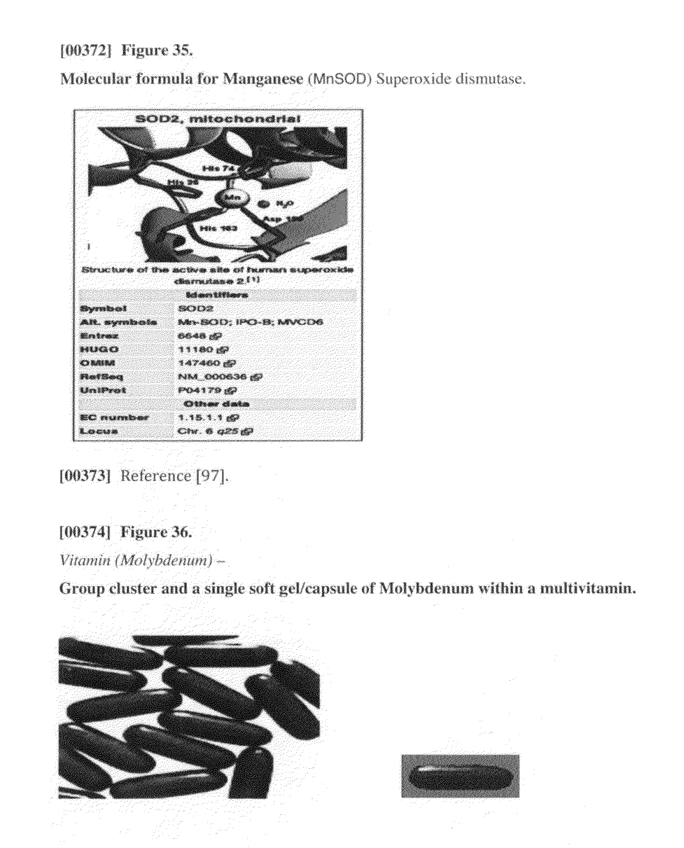

Manganese (Manganese Superoxide dismutase) (MnSOD) (Referring to FIG. 34-35)—

Manganese is an essential trace mineral that is required in small amounts to manufacture enzymes necessary for the metabolism of proteins and fat. It also supports the immune system, blood sugar balance, and is involved in the production of cellular energy, reproduction and bone growth. This micronutrient activates one or more enzymes in fatty acid synthesis. Manganese also activates the enzymes responsible for DNA and RNA production. Closely associated with copper and zinc, manganese also participates directly in the photosynthetic creation of oxygen from water. Manganese acts as a catalyst and cofactor in many enzymatic processes involved in the synthesis of fatty acids and cholesterol. Manganese aids in the formation of connective tissue, bones, blood-clotting factors, and sex hormones and plays a role in fat and carbohydrate metabolism, calcium absorption, and blood sugar regulation. [95]

Manganese activates enzyme systems involved in protein and energy metabolism. Manganese is involved in connective tissue and bone formation, insulin action, and cholesterol synthesis. Manganese is vital in the biosynthesis of collagen as it helps activate key enzymes in the formation of collagen. Manganese is a component of the antioxidant enzyme manganese superoxide dismutase (MnSOD). Antioxidants such as MnSOD can neutralize free radicals and may reduce or even help prevent some of the damage they cause. Manganese may be found in whole grains, cereals, fruits, vegetables, and tea. The total amount of manganese in the adult human is about 10-20 mg, which is concentrated mainly in the liver, skeleton, pancreas, and brain. [95]

Superoxide dismutases are a class of enzymes that catalyze the dismutation of superoxide into oxygen and hydrogen peroxide. As such, they are an important antioxidant defense in nearly all cells exposed to oxygen. [96]

Manganese ions are able to scavenge hydroxyl and superoxide radicals. Manganese is a crucial component of the metalloenzyme manganese superoxide dismutase (MnSOD). Manganese superoxide dismutase (MnSOD) is the principal antioxidant enzyme of mitochondria and is the principal constituent of the mitochondrial oxidant defense system. Manganese is an important cofactor in the enzymes necessary for mucopolysaccharide synthesis. Mucopolysaccharide is an important constituent in both skeletal and cartilage structural matrix. Manganese is essential for the enzymatic incorporation of xylose and galactose in glycoproteins. Manganese is also involved in the transfer of high-energy phosphate groups by phosphotransferase enzymes. [97]

Rich dietary sources of manganese include nuts and seeds, wheat germ and whole grains, legumes, and pineapples. Several forms of manganese are found in supplements, including manganese gluconate, manganese sulfate, manganese ascorbate, and amino acid chelates of manganese (aspartate, picolinate, fumarate, malate, succinate, citrate, and amino acid chelate). [95]

Daily Amount of Molybdenum—dosage of about 0.45 mcg 90 mcg per day.

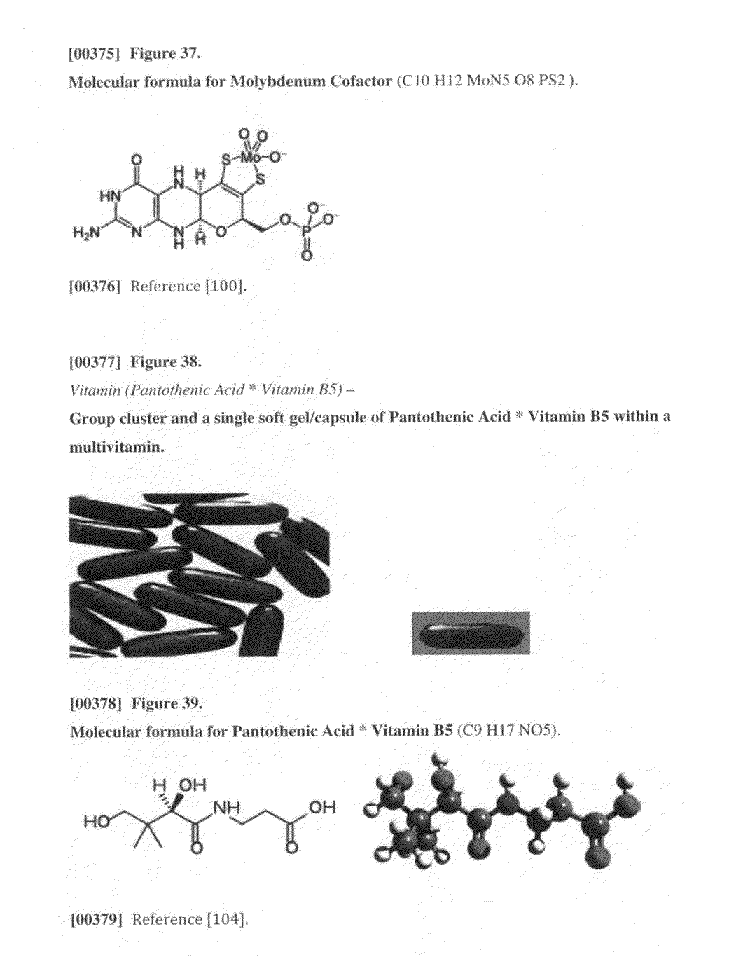

Molybdenum (C10 H12 MoN5 O8 PS2) (Referring to FIG. 36-37)—

Molybdenum cofactor is a cofactor required for the activity of enzymes such as sulfite oxidase, xanthine oxidoreductase, and aldehyde oxidase. [98] [99]

Molybdenum is a trace element considered a micronutrient, meaning a nutrient needed in very small amounts. Almost every living organism requires it. It is a cofactor for enzymes that carry out important chemical transformations in the global carbon, nitrogen, and sulfur cycles. Thus, molybdenum-dependent enzymes are not only required for the health of people, but also for the health of ecosystems. [101]

Molybdenum is an essential trace mineral considered essential in human nutrition. This is because, as tiny as the required amounts are, the consequences of their absence (deficiency) are severe. The active biological form of molybdenum is known as the molybdenum cofactor. It is found in several tissues of the human body and is required for the activity of enzymes that are involved in eliminating toxic substances, including the catabolism of purines, which produces uric acid, formed primarily in the liver and excreted by the kidney into the urine. [101]

Following absorption, molybdenum is transported by the blood to the liver and to other tissues of the body. In the molybdate form, it is carried in the blood bound to alpha-macroglobulin and by adsorption to red blood cells. The liver and kidney store the highest amounts of molybdenum. The molybdenum cofactor is made in cells and consists of a molybdenum atom bound to tricyclic pyranopterin molecules, the simplest of which is known as molybdopterin.

Molybdenum is involved in the pathways of purine degradation and formation of uric acid. In some animals, adding a small amount of dietary molybdenum enhances growth. In human, molybdenum forms oxides and is a component of a pterin coenzyme essential for the activity of xanthine oxidase, sulfite oxidase, and aldehyde oxidase.

Molybdenum is a component of several important interactions that lead to detoxification of the liver. Molybdenum is concentrated primarily in the liver, kidney, bone, and skin. Molybdenum absorption occurs readily in gastrointestinal tract, and excretion occurs primarily via the urine. Molybdenum is involved in breaking down certain amino acids (the building blocks of protein) and the production of waste products for excretion in the urine. It is involved in the chemical reactions that form bone, cartilage and blood. [102]

Beans, beef liver, cereal grains, dark green leafy vegetables, legumes, and peas are all good sources of molybdenum.

Daily Amount of Pantothenic Acid* Vitamin B5—dosage of about 0.1 mg 20 mg per day.

Pantothenic Acid (C9 H17 NO5) (Referring to FIGS. 38-39)—[3-[(2,4-dihydroxy-3,3-dimethylbutanoyl)amino]propanoic acid]

Roger J. Williams discovered Pantothenic acid in 1919. [103]

Pantothenic acid, also called pantothenate or vitamin B5, is a water-soluble vitamin.

For many animals, pantothenic acid is an essential nutrient. Animals require pantothenic acid to synthesize coenzyme-A (CoA), as well as to synthesize and metabolize proteins, carbohydrates, and fats. [105]

Since pantothenic acid participates in a wide array of key biological roles, it is essential to all forms of life. [108] As such, deficiencies in pantothenic acid may have numerous wide-ranging effects.

Pantothenic acid is therefore important for the maintenance and repair of all cells and tissues. Coenzyme A is involved in reactions that supply energy, in the synthesis of essential lipids, sterols (e.g. cholesterol), hormones (e.g. growth, stress and sex hormones), neurotransmitters, porphyrin (the oxygen-carrying red blood cell pigment) and antibodies, and in the metabolism of drugs and in alcohol detoxification. Another essential role of pantothenic acid concerns acyl carrier protein, an enzyme involved in the synthesis of fatty acids. In the process of fat burning, pantothenic acid works in concert with coenyzme Q10 and L-carnitine. [106]

Pantothenic acid in the form of CoA is also required for acylation and acetylation, which, for example, are involved in signal transduction and enzyme activation and deactivation, respectively. [107]

When in foods, most pantothenic acid is in the form of CoA or acyl carrier protein (ACP). For the intestinal cells to absorb this vitamin, it must be converted into free pantothenic acid. [109]

Free pantothenic acid is absorbed into intestinal cells via a saturable, sodium-dependent active transport system. [110]

Symptoms of deficiency are similar to other vitamin B deficiencies. There is impaired energy production, due to low CoA levels, which could cause symptoms of irritability, fatigue, and apathy. Deficiency in pantothenic acid can also cause hypoglycemia, or an increased sensitivity to insulin. [109]

Small quantities of pantothenic acid are found in nearly every food, with high amounts in whole-grain cereals, legumes, eggs, meat, royal jelly, avocado, and yogurt. [111] It is commonly found as its alcohol analog, the pro-vitamin panthenol, and as calcium pantothenate. Pantothenic acid is an ingredient in some hair and skin care products.

Daily Amount of Saw Palmetto—dosage of about 1.6 mg 320 mg per day.

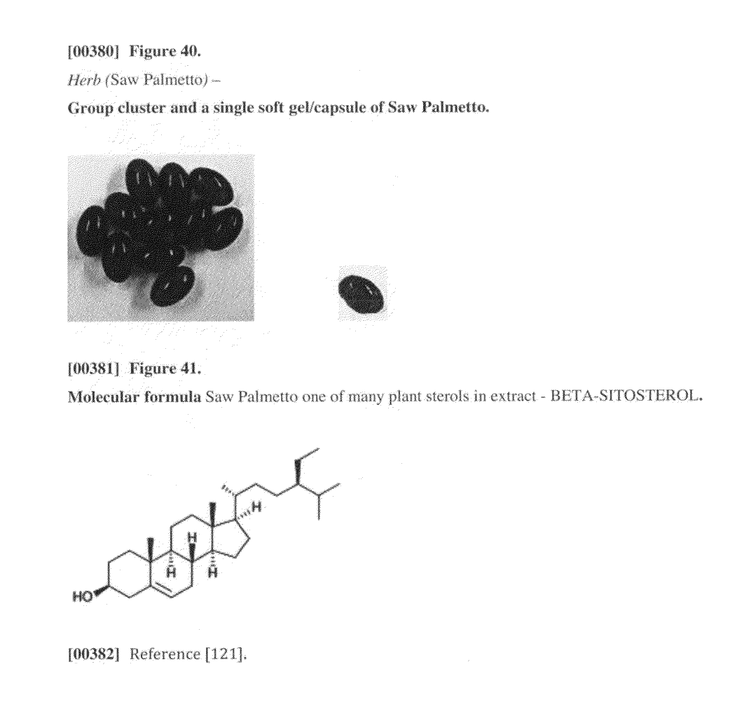

Saw Palmetto (C18 H34 O2) (Referring to FIG. 40-41)—[Sabal serrulatum, Palmae]

Saw palmetto extract is an extract of the fruit of Serenoa repens. It is rich in fatty acids and phytosterols. Saw palmetto is used in several forms of traditional medicine. Aboriginal Americans used it for food and in the treatment of a variety of urinary and reproductive system problems. [112]

In the United States, the medicinal uses of saw palmetto were first documented in 1879 by Dr. J. B. Read, a physician in Savannah, Ga., who published a paper on the medicinal benefits of the herb in the April 1879 issue of American Journal of Pharmacy. He found the herb useful in treating a wide range of conditions. “By its peculiar soothing power on the mucous membrane it induces sleep, relieves the most troublesome coughs, promotes expectoration, improves digestion, and increases fat, flesh and strength. Its sedative and diuretic properties are remarkable,” Read wrote. “Considering the great and diversified power of the saw palmetto as a therapeutic agent, it seems strange that it should have so long escaped the notice of the medical profession.” [113]

There are a variety of compounds within Saw palmetto Fruit Extract (Saw palmetto P.E.). As a rule they are divided into four major categories:
1) Free fatty acids. The ones in highest concentration include oleic acid, lauric acid, myristic acid and palmitic acid.
2) Phytosterols (plant sterols). These plant sterols have a chemical structure similar to cholesterol. The most commonly found phytosterols in Saw palmetto Fruit Extract (Saw palmetto P.E.) are beta-sitosterol, campesterol, stigmasterol and cycloartenol.
3) Free fatty alcohols. Are fatty acids joined to an alcohol molecule.
4) Monoglycerides, are single fatty acids attached to a three-carbon glycerol molecule. [114]

Inhibition of both forms of 5-alpha-reductase with no reduction in cellular capacity to secrete prostate-specific antigen is indicated. [115] [116] [117] [118]

In the latest Cochrane Database review. (2009) “The evidence suggests that Serenoa repens provides mild to moderate improvement in urinary symptoms and flow measures.” [119]

Research has shown that saw palmetto inhibits one of the active forms of testosterone in the body (dihydrotestosterone) from stimulating cellular reproduction in the prostate gland. Saw palmetto inhibits testosterone from binding to and thereby stimulating prostate cells. This serves to reduce multiplication of prostatic cells and reduces prostatic enlargement.

Saw palmetto is recommended to treat weakening urinary organs and the resulting incontinence that may occur in elderly people or women after menopause. Saw palmetto strengthens the urinary organs and has been recommended for kidney stones. [120]

Daily Amount of Selenium—dosage of about 0.2 mcg 40 mcg per day.

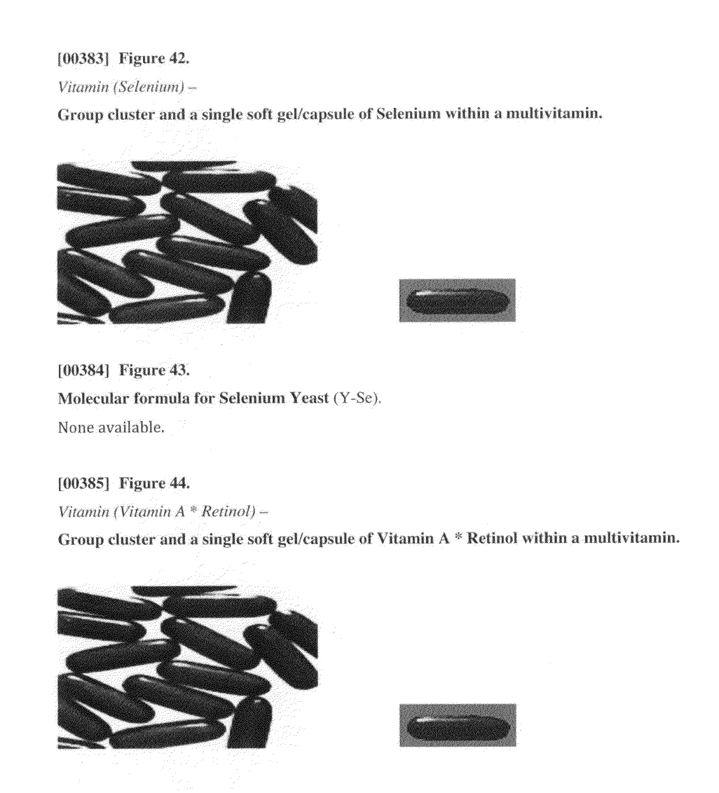

Selenium Yeast (Y—Se) (Referring to FIG. 42-43)—Selenium yeast is a dietary supplement that is used to increase selenium in your diet. [123]

Selenium is an essential trace mineral that is a component of major antioxidant enzymes, selenoproteins (glutathione peroxidase, thioredoxin reductase). Glutathione peroxidase is responsible for detoxification in the body by reducing peroxide free radicals that include lipid peroxide formation in cell membranes. Reduction of peroxides formed by oxidation of membrane phospholipids breaks the auto-oxidative chain reaction that damages cell membranes. L-selenomethionine is, an organic form of selenium, accumulates in the body because it is incorporated into proteins in place of the amino acid methionine. It is stored in the body and is slowly released. [124]

The body uses the nutrient selenium provided by 1-selenomethionine to make antioxidant proteins, such as glutathione peroxidase and thioredexin reductase, which protect against cancer-causing free radicals. A byproduct of 1-selenomethionine, methylselenol, is probably the anti-cancer form of selenium. Selenium is also essential for normal functioning of the immune system and thyroid gland. [124]

Selenium is incorporated into proteins to make selenoproteins, which are important antioxidant enzymes. At least 25 selenoproteins have been identified in human biochemistry. The major selenoproteins include glutathione peroxidases, iodothyronine deiodinases, thioredoxin reductases, selenoprotein P, selenoprotein W and selenophosphate synthetase. Glutathione peroxidase neutralizes hydrogen peroxide, which is produced by some cell processes and would otherwise damage cell membranes. Thioredoxin reductase, another selenoenzyme that acts as an antioxidant, is responsible for degrading peroxides and hydroperoxides outside cell membranes. Peroxides and hydroperoxides have been shown to cause cell death, DNA damage, and tissue atrophy. [124]

Selenium along with other minerals can help build up white blood cells, enhancing the body's ability to fight illness and infection. The antioxidant properties of selenoproteins help prevent cellular damage from free radicals. Free radicals are natural by-products of oxygen metabolism that may contribute to the development of chronic diseases such as cancer and heart disease. Selenium helps repair cells in the lungs and other organs damaged by oxidative stress, it promotes healthy immune system function, and protects red blood cells and cell membranes. Selenium may help prevent the two most common causes of impaired vision and blindness in older people, cataracts and macular degeneration, by providing antioxidant actions that fight free radicals. [124]

Selenium also acts as an anti-inflammatory agent in the treatments of arthritis and other autoimmune diseases. Selenium inhibits prostaglandins, which cause inflammatory reactions in the body. Selenium's antioxidant and anti-inflammatory actions may be enhanced when combined with vitamin E. [124] Selenium also plays a role in the functioning of the thyroid gland and in every cell that uses thyroid hormone, by participating as a cofactor for the three known thyroid hormone deiodinases, which activate and then deactivate its hormones. [124]

Daily Amount of Vitamin A* Retinol—dosage of about 25 IU 5000 IU per day.

Vitamin A* Retinol (C20 H30 O) (Referring to FIGS. 44-45)—[2E,4E,6E,8E)-3,7-Dimethyl-9-(2,6,6-trimethyl-1-cyclohexen-1-yl)-2,4,6,8-nonatetraen-1-ol (Retinol)]

Vitamin A (or Vitamin A Retinol) is a vitamin that is needed by the retina of the eye in the form of a specific metabolite, the light-absorbing molecule retinal, that is absolutely necessary for both low-light (scotopic vision) and color vision. Vitamin A also functions in a very different role, as an irreversibly oxidized form of retinol known as retinoic acid, which is an important hormone-like growth factor for epithelial and other cells. [126]

Receptor cells in the retina of the eye (rod cells) contain a light-sensitive pigment called rhodopsin, which is a complex of the protein opsin and the vitamin A metabolite retinal. The light-induced disintegration of the pigment triggers a series of events that generate an electrical signal to the optic nerve. Rhodopsin can only be regenerated from opsin and vitamin A. Rod cells with this pigment can detect very small amounts of light, making them important for night vision. [127]

Vitamin A is structurally related to carotene. Carotene is converted into vitamin A in the liver, two molecules of Vitamin A are formed from one molecule of beta-carotene. Vitamin A is manufactured by extraction from fish-liver oil and by synthesis from beta-ionone. Vitamin A is carried through the body by fat. The body can store this type of vitamin in fat tissue.

During the absorption process in the intestines, retinol is incorporated into chylomicrons as the ester form, and it is these particles that mediate transport to the liver. Storage of vitamin A in liver cells (hepatocytes) is via the ester derivative, but when retinol is needed in other tissues, it is de-esterified and released into the blood as the alcohol. Retinol then attaches to a serum carrier, retinol-binding protein, for transport to target tissues. A binding protein inside cells, cellular retinoic acid binding protein, serves to store and move retinoic acid intracellular. [128]

Retinoic acid combines with specific nuclear receptor proteins that bind to DNA and regulate the expression of various genes, thereby influencing numerous physiological processes. Retinoic acid is therefore classified as a hormone. Retinoic acid plays an important role in reproduction and embryonic development, particularly in the development of the spinal cord and vertebrae, limbs, heart, eyes and ears, and maintaining normal skin health.

Vitamin A is required for the normal functioning of the immune system and therefore helps to protect against infections in a number of ways. It is essential in maintaining the integrity and function of the skin and mucosal cells, which function as a mechanical barrier and defend the body against infection. Vitamin A also plays a central role in the development and differentiation of white blood cells, such as lymphocytes, killer cells and phagocytes, which play a critical role in the defense of the body against pathogens.

Daily Amount of Vitamin B1* Thiamine—dosage of about 0.02 mg 4 mg per day.

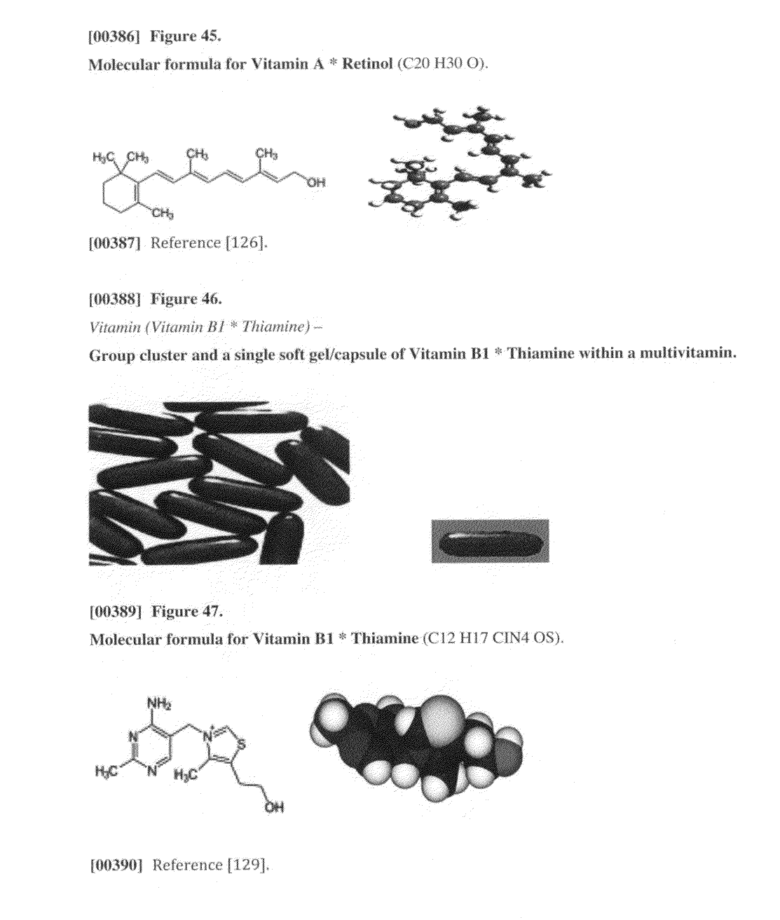

Vitamin B1* Thiamine (C12 H17 CIN4 OS) (Referring to FIGS. 46-47)—[2-[3-[(4-Amino-2-methyl-pyrimidin-5-yl)methyl]-4-methyl-thiazol-5-yl]ethanol

Thiamine was the first of the water-soluble vitamins to be described, [130] leading to the discovery of more such trace compounds essential for survival and to the notion of vitamin.

Thiamine is found in a wide variety of foods at low concentrations. Yeast, yeast extract and pork are the most highly concentrated sources of thiamine. In general, cereal grains are the most important dietary sources of thiamine, by virtue of their ubiquity. Of these, whole grains contain more thiamine than refined grains, as thiamine is found mostly in the outer layers of the grain and in the germ (which are removed during the refining process).

Thiamin promotes good circulation and supports normal cognitive brain function. It also functions as an antioxidant, guarding the body from the damaging effects of free radicals. Thiamin deficiency can happen due to a number of factors, including severe dieting, alcoholism, liver disease, and kidney dialysis. [131]

The main functions of thiamin are connected to its role as a coenzyme in the form of thiamin pyrophoshate (TPP). Coenzymes are ‘helper molecules’ which activate enzymes, the proteins that control the thousands of biochemical processes occurring in the body. TPP acts as a “helper molecule” in about 25 enzymatic reactions and plays an essential role in the production of energy from food in the carbohydrate metabolism as well as in the links between carbohydrate, protein and fat metabolism. It is one of the key compounds for several reactions in the breakdown of glucose to energy. Furthermore, TPP is coenzyme for the metabolism of branched-chain keto acids that are derived from branched-chain amino acids. [132]

Another important function of thiamin is its activation of an enzyme called “transketolase”, which in turn catalyzes reactions in the pentose phosphate pathway. This pathway is the basis for the production of many prominent compounds, such as ATP, GTP, NADPH and the nucleic acids DNA and RNA. Certain non-coenzyme functions of thiamin are important for nerve tissues and muscles. Its triphosphate form (TTP) in particular plays a role in the conduction of nerve impulses, in the metabolism of the neurotransmitters acetylcholine, adrenalin, and serotonin and in aerobic metabolism. [132]

Thiamine is essential for the transmission of certain types of nerve signal between the brain and the spinal cord. Depression, poor memory, muscle weakness and stiffness, nerve tingling, burning sensation and numbness, tiredness, headache, loss of appetite and nausea are some of the symptoms and signs of its deficiency. [131]

Daily Amount of Vitamin B2* Riboflavin—dosage of about 0.02 mg 4 mg per day.

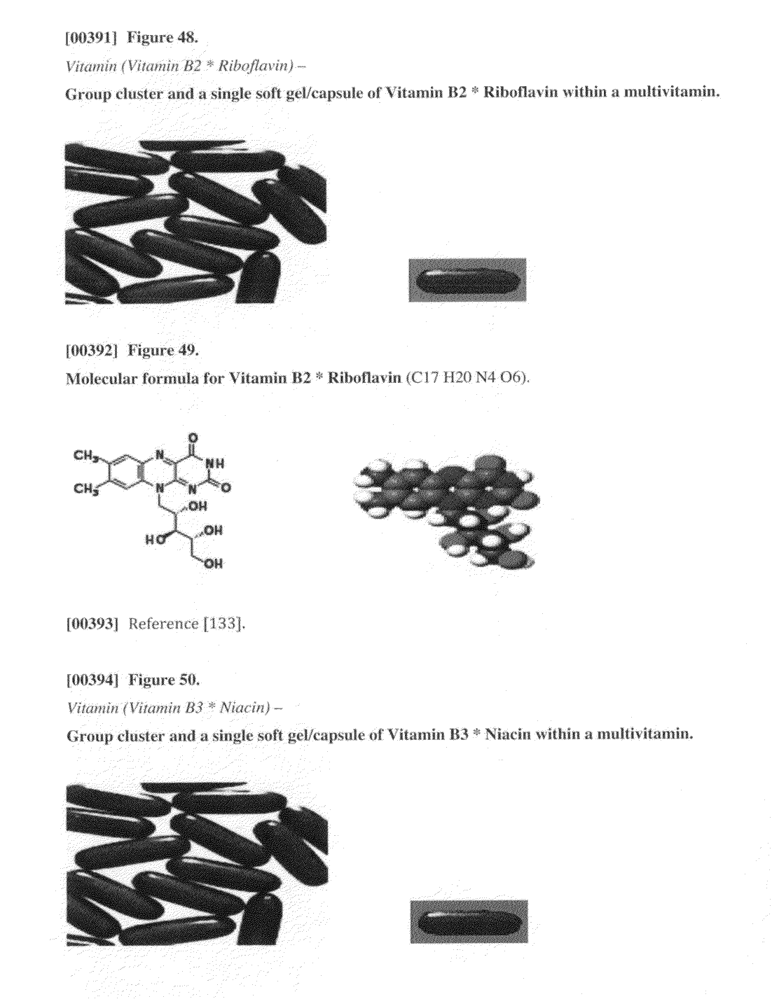

Vitamin B2* Riboflavin (C17 H20 N4 O6). (Referring to FIGS. 48-49)—[7,8-dimethyl-10-[(2R,3R,4S)-2,3,4,5-tetrahydroxypentyl]benzo[g]pteridine-2,4(3H,10H)-dione]

Riboflavin, also known as vitamin B2 is an easily absorbed micronutrient with a key role in maintaining health in humans and animals. It is the central component of the cofactors FAD and FMN, and is therefore required by all flavoproteins. As such, vitamin B2 is required for a wide variety of cellular processes. It plays a key role in energy metabolism, and for the metabolism of fats, ketone bodies, carbohydrates, and proteins. [133]

Vitamin B2 (riboflavin) is a critical component of metabolizing carbohydrates and turning them into sugar, which the body then uses for energy. It is necessary for the normal growth and development of tissues—particularly skin, hair, and other connective tissues. Vitamin B2 is also needed for the production of antibodies and for healthy immune system function. A deficiency of B2 can have a severe impact on the metabolism of carbohydrates, fats, and protein into energy.

Flavin coenzymes are essential for energy production via the respiratory chain, as they act as catalysts in the transfer of electrons in numerous essential oxidation-reduction reactions (redox reactions). Riboflavin coenzymes are also essential for the conversion of pyridoxine (vitamin B6) and folic acid into their coenzyme forms and for the transformation of tryptophan to niacin. Vitamin B2 also promotes normal growth and assists in the synthesis of steroids, red blood cells, and glycogen. Furthermore, it helps to maintain the integrity of mucous membranes, eyes, and the nervous system, and is involved in the production of adrenaline by the adrenal glands. [134]

Vitamin B2 helps prevent and is used to treat migraine headaches, cataracts, rheumatoid arthritis, and a number of skin disorders such as acne, dermatitis, and eczema. In the treatment of anemia, adding Vitamin B2 to iron supplements has shown to increase its effectiveness. Vital to maintaining a proper metabolism, riboflavin also helps to shore up the immune system by reinforcing antibody reserves, the body's first line of defense against infection. Along with iron, riboflavin is essential for producing the red blood cells that carry oxygen throughout the body. The body uses extra riboflavin to keep tissue in good repair and speed healing of wounds, burns and other injuries. [135]

Riboflavin deficiency has profound effects on the metabolism of carbohydrates, fats, and protein. All three of these basic food elements require riboflavin if they are to be properly utilized by the body. A deficiency of vitamin B2 (riboflavin) may result in bloodshot eyes, inflammation in the mouth, a sore and burning tongue, and cracks on the lips and in the corners of the mouth. It may also result in dull or oily hair, an oily skin, premature wrinkles on the face and arms, and split nails. Riboflavin deficiency also leads to the malfunctioning of the adrenal glands. [135]

Daily Amount of Vitamin B3* Niacin—dosage of about 0.2 mg 40 mg per day.

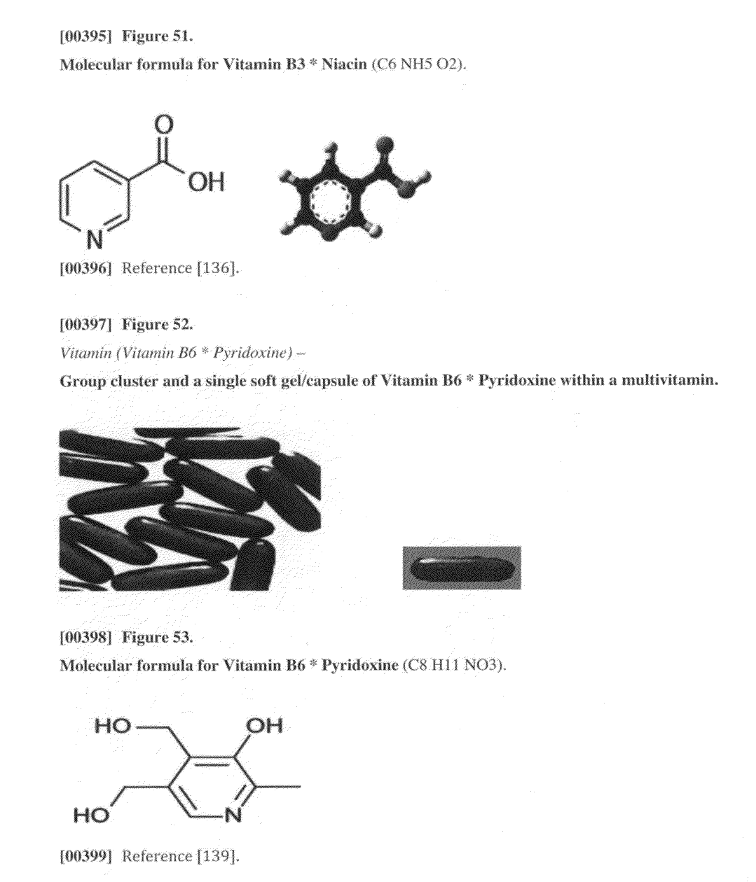

Vitamin B3* Niacin (C6 NH5 O2) (Referring to FIG. 50-51)—[Nicotinic acid]

Niacin plays an important role in ridding the body of toxic and harmful chemicals. It also helps the body make various sex and stress-related hormones in the adrenal glands and other parts of the body. Vitamin B3 is essential for the activity of many enzymes in the body. Enzymes are special substances that speed up chemical reactions in the body. These enzymes are responsible for production of energy in the body, the breakdown of dietary fats, the production of certain hormones and cholesterol, the processing of genetic material (DNA) and the growth and maturation of the cells in the body. Niacin is effective in improving circulation and reducing cholesterol levels in the blood. [137]

Niacin is a water-soluble vitamin that participates in more than 50 metabolic functions, all of which are important in the release of energy from carbohydrates. Niacin has a pivotal role in metabolic functions. It supplies vital energy and maintains integrity in body cells.

The term niacin refers to both nicotinic acid and its amide derivative, nicotinamide (niacin amide). Both are used to form the coenzymes nicotinamide adenine dinucleotide (NAD) and nicotinamide adenine dinucleotide phosphate (NADP). Niacin is a member of the water-soluble B-vitamin complex. The amino acid tryptophan can be converted to nicotinic acid in humans, therefore niacin is not really a vitamin provided that an adequate dietary supply of tryptophan is available.

The coenzymes NAD and NADP are required for many biological oxidation-reduction (redox) reactions. About 200 enzymes require NAD or NADP. NAD is mainly involved in reactions that generate energy in tissues by the biochemical degradation of carbohydrates, fats and proteins. NADP functions in reductive biosynthesis such as the synthesis of fatty acids and cholesterol.

NAD is also required as a substrate for non-redox reactions. It is the source of adenosine diphosphate (ADP)-ribose, which is transferred to proteins by different enzymes. These enzymes and their products seem to be involved in DNA replication, DNA repair, cell differentiation and cellular signal transduction. [138]

It can help to relieve negative personality behavior such as schizophrenia, depression, delusions, and dementia. Niacin can also help relieve acne, migraines, vertigo, forgetfulness, high blood pressure and diarrhea. Niacin, in synergy with chromium improves blood sugar regulation by helping insulin function. Niacin has helped elderly patients regain mental clarity; because it dilates blood vessels, niacin brings more oxygen to the brain. Niacin has helped insomniacs because of its sleep-inducing qualities. Niacin helps stabilize blood sugar, and has been used to treat acne. Niacin has also been extremely beneficial to arthritis sufferers. Dietary vitamin B3, along with other nutrients is important for normal vision and prevention of cataracts. [138]

Daily Amount of Vitamin B6* Pyridoxine—dosage of about 0.02 mg 4 mg per day.

Vitamin B6* Pyridoxine (C8 H11 NO3). (Referring to FIGS. 52-53)—[4,5-Bis(hydroxymethyl)-2-methylpyridin-3-ol]

Vitamin B6, also known as pyridoxine, is a water-soluble B vitamin. Vitamin B6 actually describes several different compounds, including pyridoxine, pyridoxal, and pyridoxamine.

Vitamin B6 (pyridoxine) acts as a coenzyme and plays a role in the metabolization of protein and carbohydrates into energy. It is also required for the production of insulin, as well as both red and white blood cells. Pyridoxine is particularly vital for keeping nerve and muscle cells healthy. Vitamin B6 is needed for the creation of DNA and RNA. Risk of vitamin B6 deficiency is quite rare, because the vitamin is contained in the majority of foods. A deficiency most often results from poor absorption of nutrients via the intestines (as in the case of chronic alcoholism or chronic diarrhea).

Vitamin B6 serves as a coenzyme of approximately 100 enzymes that catalyze essential chemical reactions in the human body. It plays an important role in protein, carbohydrate and lipid metabolism. Its major function is the production of serotonin from the amino acid tryptophan in the brain and other neurotransmitters, and so it has a role in the regulation of mental processes and mood. Furthermore, it is involved in the conversion of tryptophan to the vitamin niacin, the formation of hemoglobin and the growth of red blood cells, the absorption of vitamin B12, the production of prostaglandins and hydrochloric acid in the gastrointestinal tract, the sodium-potassium balance, and in histamine metabolism. As part of the vitamin B-complex it may also be involved in the down-regulation of the homocysteine blood level. Vitamin B6 also plays a role in the improvement of the immune system. [140]

The forming of histamine, serotonin, dopamine and adrenaline are dependent on vitamin B6. Vitamin B6 is required for the production of serotonin and helps to maintain healthy immune system functions, to protect the heart from cholesterol deposits, and to prevent kidney stone formation. [141]

Vitamin B6 is indicated for the treatment of sideroblast anemia, neurologic disturbances, seborrhoeic dermatitis, and cheilosis. In combination with folic acid and vitamin B12, vitamin B6 lowers homocysteine levels that are an amino acid linked to heart disease and stroke, and possibly other diseases as well, such as osteoporosis, and Alzheimer's disease. Pyridoxine is required for the balancing of hormonal changes in women as well as assisting the immune system and the growth of new cells. Vitamin B6 promotes iron excretion and this has been used as a rationale for treatment in iron storage diseases. [141]

All three forms of vitamin B6 (pyridoxine, pyridoxal and pyridoxamine) are readily absorbed in the small intestine by an energy dependent process. All three are converted to pyridoxal phosphate in the liver, a process that requires zinc and riboflavin.

Daily Amount of Vitamin B9* Folic Acid—dosage of about 4 mcg 800 mcg per day.

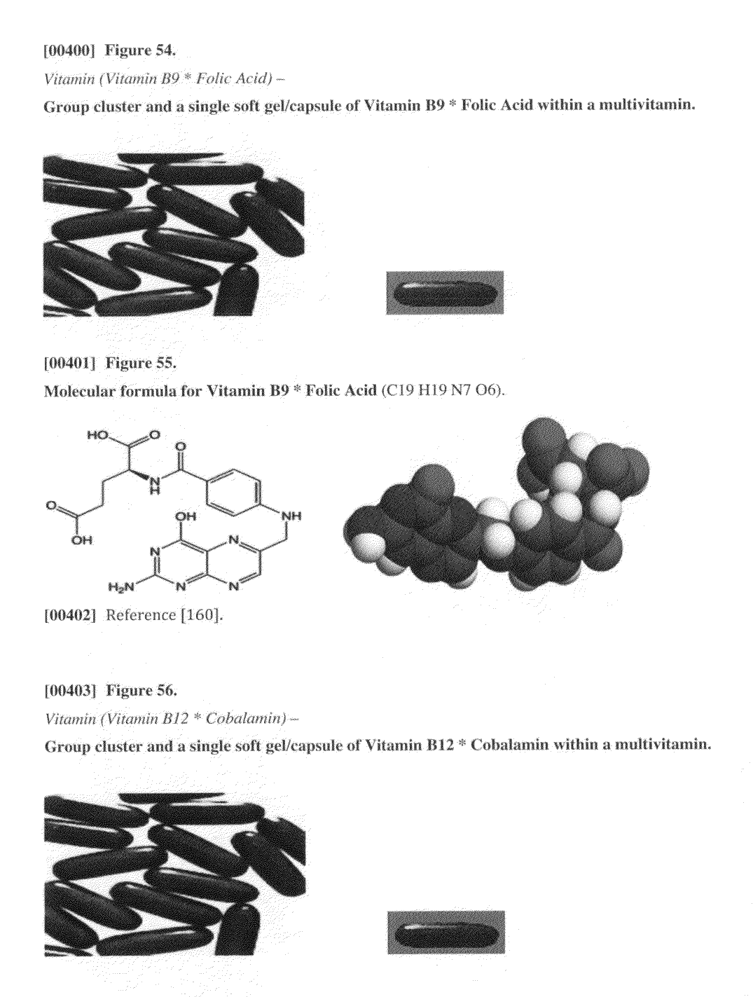

Vitamin B9* Folic Acid (C19 H19 N7 O6) (Referring to FIGS. 54-55)—[(2S)-2-[(4-{[(2-amino-4-hydroxypteridin-6-yl)methyl]amino}phenyl)formamido]pentanedioic acid]

Folic acid (also known as vitamin B9) [161] and folate (the naturally occurring form), are forms of the water-soluble vitamin B₉. Folic acid is itself not biologically active, but its biological importance is due to tetrahydrofolate after its conversion to dihydrofolic acid in the liver. [160]

Vitamin B₉ (folic acid and folate inclusive) is essential to numerous bodily functions. The human body needs folate to synthesize DNA, repair DNA, and methylate DNA as well as to act as a cofactor in biological reactions involving folate. [163] It is especially important in aiding rapid cell division and growth, such as in infancy and pregnancy, as well as in “feeding” some cancers. While a normal diet also high in natural folates may decrease the risk of cancer, there is diverse evidence that high folate intake from supplementation may actually promote some cancers as well as precancerous tumors and lesions. Children and adults both require folic acid to produce healthy red blood cells and prevent anemia. [164] Folates are therefore essential for normal cell division, proper growth and for optimal functioning of the bone marrow. [162]

Folate deficiency is one of the commonest vitamin deficiencies. It can result from inadequate intake, defective absorption, abnormal metabolism or increased requirements. Diagnosis of a subclinical deficiency relies on demonstrating reduced red cell folate concentration or on other biochemical evidence such as increased homocysteine concentration, since haematological manifestations are usually absent. Early symptoms of folate deficiency are non-specific and may include tiredness, irritability and loss of appetite.

Severe folate deficiency leads to megaloblastic anaemia, a condition in which the bone marrow produces giant, immature red blood cells. At an advanced stage of anaemia symptoms of weakness, fatigue, irritability, headache, and palpitations appear. If left untreated, megaloblastic anaemia may be fatal. Gastrointestinal symptoms also result from severe folate deficiency. [162]

Folate is destroyed by exposure to ultraviolet radiation (UVR), which puts the skin under increased demands for optimal folate intake. A 2010 cell culture study showed that folic acid supplementation may positively influence skin health. [165]

A number of different observational studies have found poor folate status to be associated with increased cancer risk. There is evidence that folate plays a role in preventing colorectal cancer. The results of two large epidemiological investigations suggest that increased folate intake may reduce breast cancer risk associated with regular alcohol consumption. Low folate levels have also been associated with Alzheimer's disease, dementia and depression. [162]

Daily Amount of Vitamin B12* Cobalamin—dosage of about 0.06 mcg 12 mcg per day.

Vitamin B12* Cobalamin (C63 H88 CoN14 O14 P) (Referring to FIG. 56-57)—[α-(5,6-dimethylbenzimidazolyl)cobamidcyanide]

Vitamin B12, also called Cobalamin, is a water-soluble vitamin with a key role in the normal functioning of the brain and nervous system, and for the formation of blood.

Cyanocobalamin is the principal form of the vitamin used for fortification of foods and in nutritional supplements. Vitamin B12's primary roles are aiding in the production of red blood cells, and in helping to maintain the health of the central nervous system. Nerves are encased in an insulating sheath that is made of a protein known as myelin. Cobalamin is critical for maintaining this myelin sheath around nerves.

Vitamin B12 is needed for normal nerve cell activity, DNA replication, and production of the mood-affecting substance SAMe (S-adenosyl-L-methionine). Vitamin B12 also works closely together with vitamin B9 (folate) to regulate the formation of red blood cells and to help iron function better in the body. Vitamin B12 is important for the activity of certain enzymes within calls that control fat, amino acid and carbohydrate metabolism. Both vitamin B12 and the vitamin folate are essential for the production of genetic material in the body (DNA and RNA). [143]

Two metabolic reactions are dependent on vitamin B12: methionine synthase reaction with methylcobalamin and methylmalonyl CoA mutase reaction with adenosylcobalamin. In its methylcobalamin form vitamin B12 is the direct cofactor for methionine synthase, the enzyme that recycles homocysteine back to methionine. There is evidence that vitamin B12 is required in the synthesis of folate polyglutamates (active coenzymes required in the formation of nerve tissue) and in the regeneration of folic acid during red blood cell formation. Methylmalonyl CoA mutase converts 1-methylmalonyl CoA to succinyl CoA (an important reaction in lipid and carbohydrate metabolism). Adenosylcobalamin is also the coenzyme in ribonucleotide reduction (which provides building blocks for DNA synthesis). [144]

Clinical Cobalamin deficiency insufficiency is rare, but occurs more frequently in older people. Vitamin B12 deficiency affects 10-15% of individuals over the age of 60.

Deficiency of vitamin B12 leads to defective DNA synthesis in cells, which affects the growth and repair of all cells. Tissues most affected are those with the greatest rate of cell turnover, e.g. those of the hematopoietic system. This can lead to megaloblastic anemia (characterized by large and immature red blood cells) and neuropathy, with numerous symptoms including: glossitis, weakness, loss of appetite, loss of taste and smell, irritability, memory impairment, mild depression, hallucination, breathlessness (dyspnea) on exertion, tingling and numbness (paresthesia). [144]

Daily Amount of Vitamin C* L-Ascorbic Acid—dosage of about 0.6 mg 120 mg per day.

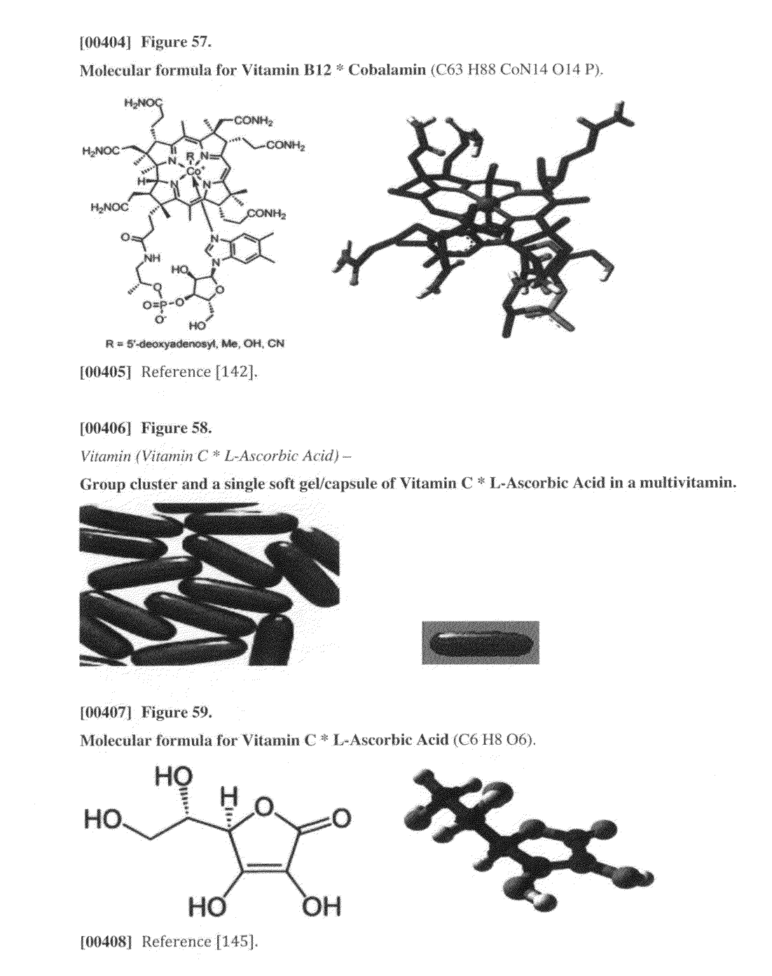

Vitamin C* L-Ascorbic Acid (C6 H8 O6). (Referring to FIGS. 58-59)—[2-oxo-L-threo-hexono-1,4- lactone-2,3-enediol]

Vitamin C is water-soluble, and probably the most famous of all the vitamins. Even before its discovery in 1932, physicians recognized that there must be a compound in citrus fruits preventing scurvy, a disease that killed as many as 2 million sailors between 1500 and 1800.

Vitamin C or L-ascorbic acid or L-ascorbate is an essential nutrient for humans and certain other animal species. In living organisms ascorbate acts as an antioxidant by protecting the body against oxidative stress. [146]

It is also a cofactor in at least eight enzymatic reactions including several collagen synthesis reactions that cause the most severe symptoms of scurvy when they are dysfunctional. [147]

The most prominent role of vitamin C is its immune stimulating effect, which is important for the defense against infections such as common colds. It also acts as an inhibitor of histamine, a compound that is released during allergic reactions. As a powerful antioxidant it can neutralize harmful free radicals and aids in neutralizing pollutants and toxins. Thus it is able to prevent the formation of potentially carcinogenic nitrosamines in the stomach. Importantly, vitamin C is also able to regenerate other antioxidants such as vitamin E. [148]

Due to these functions vitamin C, especially in combination with zinc, is important for the healing of wounds. Vitamin C contributes to the health of teeth and gums, preventing hemorrhaging and bleeding. It also improves the absorption of iron from the diet, and is needed for the metabolism of bile acids, which may have implications for blood cholesterol levels and gallstones. Also, vitamin C plays an important role in the synthesis of several important peptide hormones and neurotransmitters and carnitine. [148]

Vitamin C may protect against heart disease by reducing the stiffness of arteries and the tendency of platelets to clump together. Long-term administration of vitamin C reverses endothelial vasomotor dysfunction in patients with coronary artery disease. Under most circumstances, dietary vitamin C is adequate for protecting against the development of or consequences from cardiovascular disease. When taken with vitamin E, vitamin C helps protect LDL (“bad”) cholesterol from oxidation, thus preventing plaque buildup in coronary arteries. Individuals with high blood levels of vitamin C have significantly reduced risk of stroke. [149]

Early symptoms of vitamin C deficiency are very general and could also indicate other diseases. They include fatigue, lassitude, loss of appetite, drowsiness and insomnia, feeling run-down, irritability, low resistance to infections and petechiae (minor capillary bleeding).

Daily Amount of Vitamin E* α-Tocopherol—dosage of about 0.5 IU 100 IU per day.

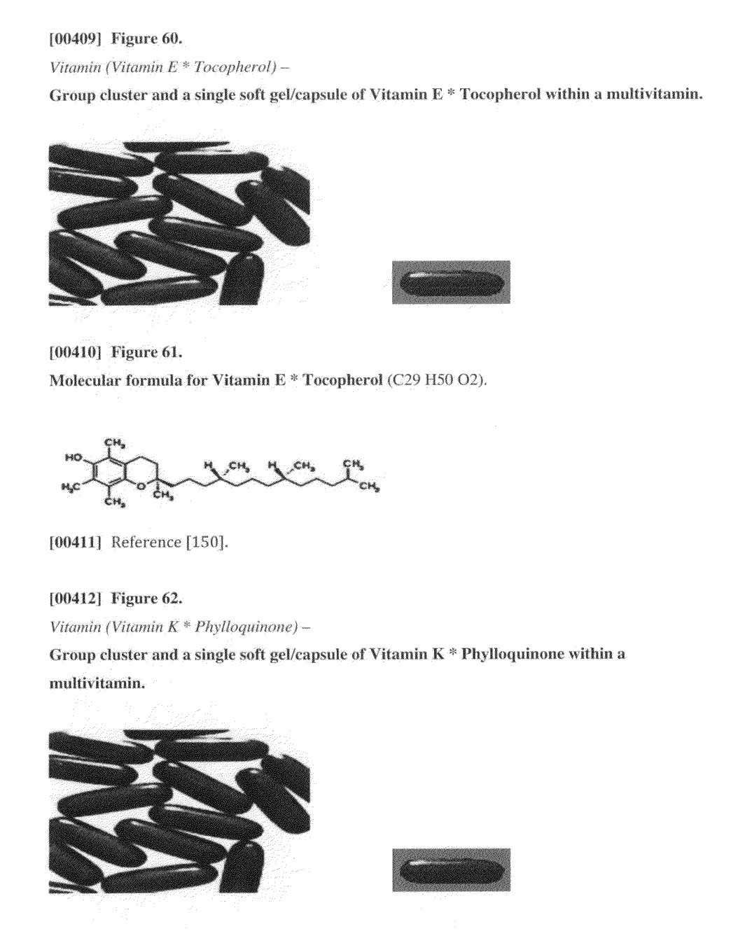

Vitamin E* α-Tocopherol (C29 H50 O2) (Referring to FIG. 60-61) [(2R)-2,5,7,8-tetramethyl-2-[(4R,8R)-4,8,12-trimethyltridecyl]-3,4-dihydro-2H-chromen-6-ol]

The term vitamin E covers eight fat-soluble compounds found in nature. Four of them are called tocopherols and the other four tocotrienols. They are identified by the prefixes α, β, γ and δ. α-Tocopherol is the most common and biologically the most active of these naturally occurring forms of vitamin E. Natural tocopherols occur in RRR-configuration only (RRR-α-tocopherol was formerly designated as d-α-tocopherol). [151]

Vitamin E is the thought to be the most effective antioxidant known to function in the body. Vitamin E actually identifies a group of fat-soluble compounds known as tocopherols and tocotrienols.

The most vital function vitamin E performs is helping to stabilize cell membranes and guarding against damage to body tissues caused by oxidation. Vitamin E acts as a powerful antioxidant that protects against damage caused by free radicals, which can contribute to cardiovascular disease and cancer. When applied to skin, vitamin E creams are thought to promote healing and protect the skin from damage.

The major biological function of vitamin E is that of a lipid soluble antioxidant preventing the propagation of free-radical reactions. Free radicals are formed in normal metabolic processes and upon exposure to exogenous toxic agents (e.g. cigarette smoke, pollutants). Vitamin E is located within the cellular membranes. It protects polyunsaturated fatty acids (PUFAs) and components of cellular membranes from oxidation by free radicals. Apart from maintaining the integrity of the cell membranes in the human body, it also protects low-density lipoprotein (LDL) from oxidation. [151]

Vitamin E as an antioxidant helps to stabilize cell membranes and protect the tissues of the skin, eyes, liver, breast, and testes, which are more sensitive to oxidation. The protective, nutritional antioxidant function of vitamin E is also performed and enhanced by other antioxidants, such as vitamin C, beta-carotene, glutathione (L-cysteine), and the mineral selenium. [152]

Vitamin E is thought to play a role in preventing atherosclerosis and cardiovascular diseases (heart disease and stroke) due to its effects on a number of steps in the development of atherosclerosis (e.g. inhibition of LDL oxidation, inhibition of smooth muscle cell proliferation, inhibition of platelet adhesion, aggregation and platelet release reaction). [151]

Vitamin E deficiency can impact the central nervous system and could result in progressive neuromuscular disorders resulting in impaired reflexes, muscular weakness, and loss of balance. A deficiency can also lead to an increased risk of cardiovascular disease and some cancers.

Daily Amount of Vitamin K* Phylloquinone—dosage about 0.4 mcg 80 mcg per day.

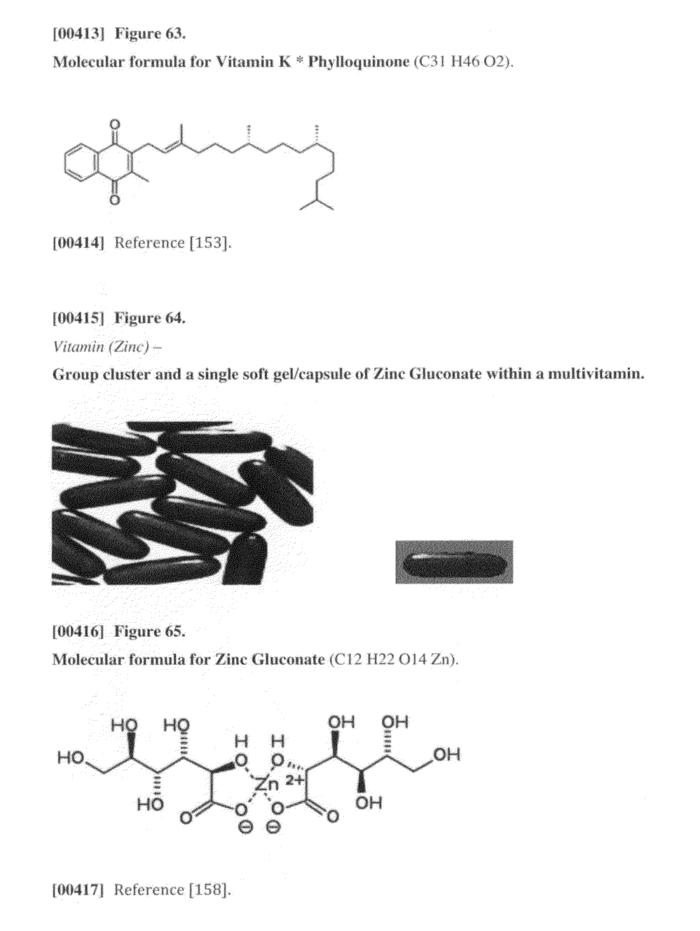

Vitamin K* Phylloquinone (C31 H46 O2) (Referring to FIG. 62-63) [2-methyl-3-[(2E)-3,7,11,15-tetramethylhexadec-2-en-1-yl] naphthoquinone]

Vitamin K plays a vital part in normal blood clotting and in forming healthy bones. Taking a vitamin K supplement could help increase bone mass in women who are post-menopausal. It also helps to prevent the calcification of body tissues, including arteries. Vitamin K may also function to help regulate blood sugar levels.

Vitamin K was identified in 1929 by Danish scientist Henrik Dam when he investigated the role of cholesterol by feeding chickens a cholesterol-depleted diet. [155]

Vitamin K is a group of structurally similar, fat soluble vitamins that are needed for the posttranslational modification of certain proteins, mostly required for blood coagulation but also involved in metabolic pathways in bone and other tissue. [153]

Vitamin K is indeed fat-soluble, and it occurs naturally in two forms: vitamin K1 (phylloquinone) is found in plants; vitamin K2 is the term for a group of compounds called menaquinones (MK-n, n being the number of isoprenyl units in the side chain of the molecule) which are synthesized by bacteria in the intestinal tract of humans and various animals. Vitamin K3 (menadione) is a synthetic compound that can be converted to K1 in the intestinal tract. It is only used in animal nutrition. [154]

Vitamin K is involved in the carboxylation of certain glutamate residues in proteins to form gamma-carboxyglutamate residues (abbreviated Gla residues). The modified residues are often (but not always) situated within specific protein domains called Gla domains. Gla residues are usually involved in binding calcium. The Gla residues are essential for the biological activity of all known Gla-proteins. [156]

Vitamin K and blood clotting—Vitamin K's primary function is to regulate normal blood clotting (due to its role in the synthesis of prothrombin). Blood clotting is a process that begins automatically when any injury produces a tear in a blood vessel. The process of blood clotting involves a collection of molecules, which circulate continuously through the bloodstream. Vitamin K regulates normal blood clotting by helping the body transport calcium. Protein Z appears to enhance the action of thrombin (the activated form of prothrombin) by promoting its association with phospholipids in cell membranes. Protein C and protein S are anticoagulant proteins that provide control and balance in the coagulation cascade. [157]

Very little vitamin K is stored in the body; small amounts of this vitamin are deposited in the liver and in the bones, but this amount is only enough to supply the body's needs for a few days.

Vitamin K prevents calcification of arteries and other soft tissue. Vitamin K may play a role in the regulation of blood sugar. The pancreas, which makes insulin, has the second highest amount of vitamin K in the body. [157]

Daily Amount of Zinc—dosage of about 0.15 mg 30 mg per day.

Zinc Gluconate (C12 H22 O14 Zn) (Referring to FIG. 64-65)—

Zinc is a mineral that is vital to many biological functions such as immune resistance, wound healing, digestion, reproduction, physical growth, diabetes control, taste and smell. The main biochemical in which zinc has been found to be necessary include: enzymes and enzymatic function, carbohydrate metabolism and protein synthesis. More than 300 enzymes in the human body require zinc for proper functioning. It is estimated that 3000 of the 100,000 or so proteins involved in human life contain zinc. Many cells secrete zinc, including the pancreas (which also secretes insulin), the salivary gland, and the prostate gland. Immune cells also secrete zinc. Zinc is an important mineral which is essential for protein synthesis and which helps to regulate the production of cells in the body's immune system. Zinc is a constituent of insulin and male reproductive fluid. Stored primarily in muscle, zinc is also found in high concentrations in red and white blood cells, the retina of the eye, bones, skin, liver, and pancreas. In men, the prostate gland stores high amounts of zinc. [159]

Zinc has a range of functions. Zinc functions as an antioxidant and is involved in many critical biochemical reactions. Zinc plays an important role as a component of many enzymes and the catalysts of enzyme systems regulating cell growth, DNA and protein synthesis, energy metabolism, regulation of gene transcription, hormone levels, and growth factor metabolism. Zinc is an important mineral which is essential for protein synthesis and which helps to regulate the production of cells in the body's immune system. [159]

As a component of many enzymes, zinc is involved in the metabolism of proteins, carbohydrates, lipids and energy. Zinc is important in the metabolism of vitamin A and collagen, cellular immunity, maintenance of taste acuity, and the development of reproductive organs.

Zinc (Zn) is contained mainly in bones, teeth, hair, skin, liver, muscle, leukocytes, and testes. Zinc is a component of several hundred enzymes, including many nicotinamide adenine dinucleotide (NADH) dehydrogenases, RNA and DNA polymerases, and DNA transcription factors as well as alkaline phosphatase, superoxide dismutase, and carbonic anhydrase.

Zinc assists in maintaining the proper concentration of vitamin E in the blood. Zinc also plays a role in the regulation of appetite, stress level, taste, and smell. It is essential for normal growth and development, and for most aspects of reproduction in both males and females. Zinc also supports normal growth and development during pregnancy, childhood, and adolescence. [159]

Signs of zinc deficiency include hair loss, skin lesions, diarrhea, wasting of body tissues, and eventually death. Eyesight, taste, smell and memory are also connected with zinc and a deficiency in zinc can cause malfunctions of these organs and functions. [159]

### EXAMPLES

### Example I

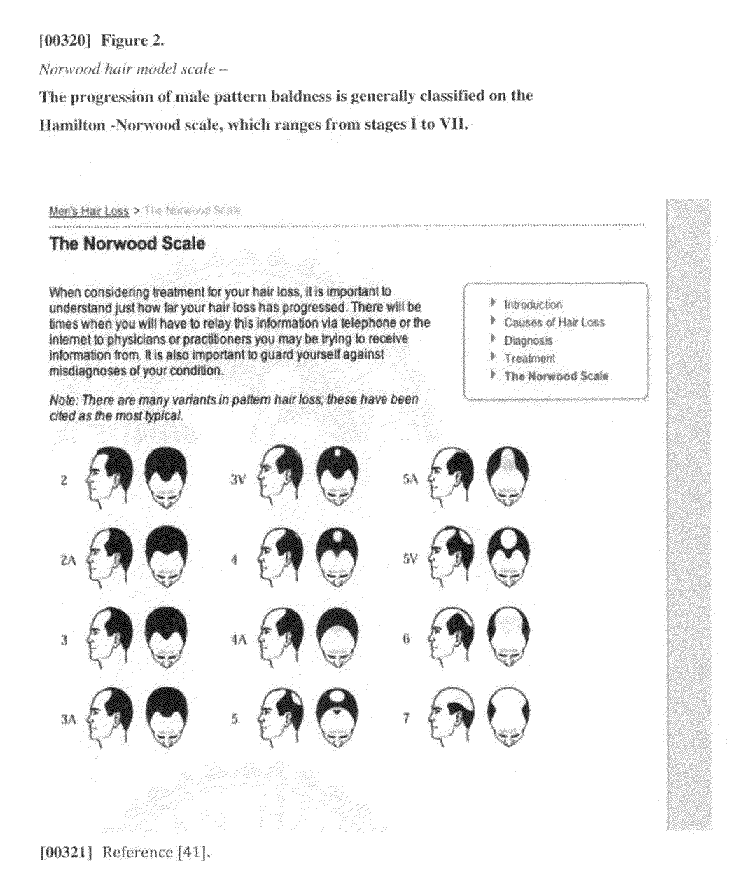

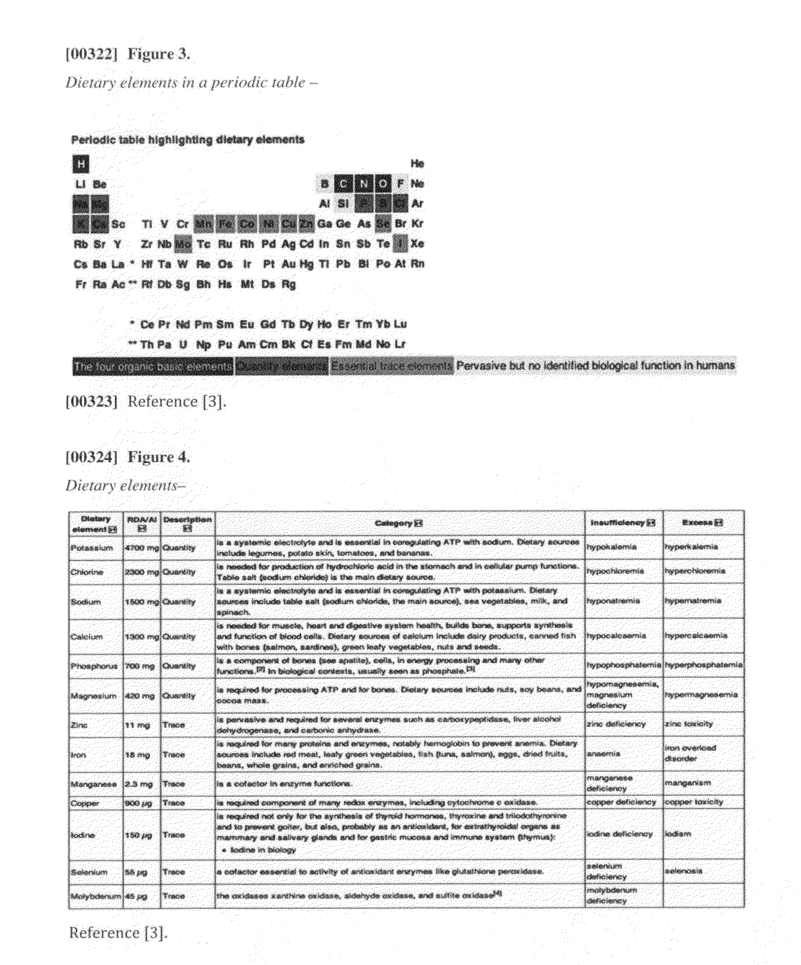

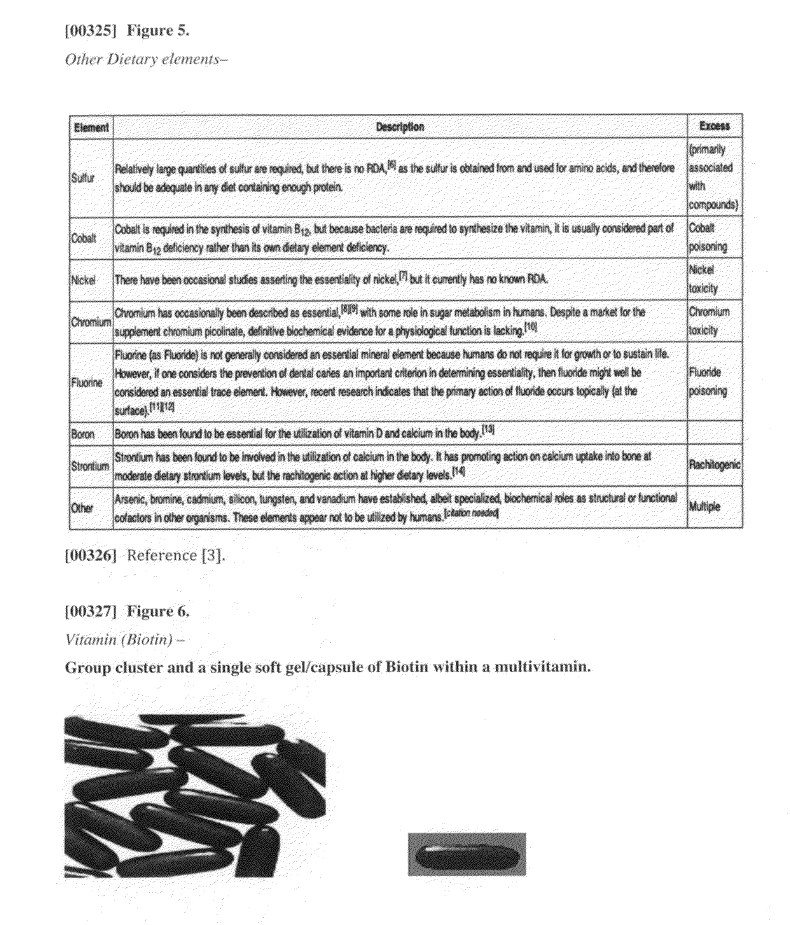

One male subject of 49 years had a complete bald area on his head for three years. The hair loss progression is detailed in the Norwood model (Referring to FIG. 2) approximately (6.5 out of 7.0). He used the said composition with correct dosage every day for seven months. In a 2-inch square section of the bald area, the subject had grown 638 new hairs.

The male subject's overall health has improved. He has more physical stamina, alertness, a tuned digestive system, less sickness with a stronger immune system, and better healing capabilities.

The composition of vitamin and herbal supplements are a mixture of Biotin, Calcium, Chromium, Copper, DHA-docosahexaenoic acid, EPA-eicosapentaenoic acid, Fish oil, Folic acid, Ginkgo biloba, Ginseng, Iodine, Iron, Magnesium, Manganese, Molybdenum, Niacin, Omega-3, Pantothenic acid, Riboflavin, Saw palmetto, Selenium, Thiamin, Vitamin A, Vitamin B12, Vitamin B6, Vitamin C, Vitamin D-cholecalciferol, Vitamin E, Vitamin K, and Zinc.

The nutritional analysis of this composition is measured and the results are listed (Referring to FIG. 66). The oral administration of said composition is at a dosage about 3749.465 mg per day for seven months.

### Example II

One female subject of 35 years used the said composition with correct dosage every day for one month. After the initial intake in a 2-week period, all nails in both hands increased growth. The initial state was about 0.0787″ or 2 mm with a growth to about 0.2559″ or 6.5 mm.

The composition of vitamin and herbal supplements are a mixture of Biotin, Calcium, Chromium, Copper, DHA-docosahexaenoic acid, EPA-eicosapentaenoic acid, Fish oil, Folic. acid, Ginkgo biloba, Ginseng, Iodine, Iron, Magnesium, Manganese, Molybdenum, Niacin, Omega-3, Pantothenic acid, Riboflavin, Saw palmetto, Selenium, Thiamin, Vitamin A, Vitamin B12, Vitamin B6, Vitamin C, Vitamin D-cholecalciferol, Vitamin E, Vitamin K, and Zinc.

The nutritional analysis of this composition is measured and the results are listed (Referring to FIG. 66). The oral administration of said composition is at a dosage about 3749.465 mg per day for one month.

### Example III

One female subject of 38 years used the said composition with correct dosage every day for one month. During this period, the subject hit her ankle against a kitchen cabinet. Later a blood-bruise blister, the size of a pencil eraser was noticed. Two days later, The skin area was healed and clear. The determined area had no trace, marks, or history of any incident.

The composition of vitamin and herbal supplements are a mixture of Biotin, Calcium, Chromium, Copper, DHA-docosahexaenoic acid, EPA-eicosapentaenoic acid, Fish oil, Folic acid, Ginkgo biloba, Ginseng, Iodine, Iron, Magnesium, Manganese, Molybdenum, Niacin, Omega-3, Pantothenic acid, Riboflavin, Saw palmetto, Selenium, Thiamin, Vitamin A, Vitamin B12, Vitamin B6, Vitamin C, Vitamin D-cholecalciferol, Vitamin E, Vitamin K, and Zinc.

The nutritional analysis of this composition is measured and the results are listed (Referring to FIG. 66). The oral administration of said composition is at a dosage about 3749.465 mg per day for one month.

### Example IV

One male subject of 42 years used the said composition with the correct dosage of one daily intake. After three daily meals, plus nine shots of liquor, the subject experienced no effects the following morning. The subject was alert with clarity. He experienced no headaches, pain, fatigue, or dizziness. The said composition had cleared all intoxication effects.

The composition of vitamin and herbal supplements are a mixture of Biotin, Calcium, Chromium, Copper, DHA-docosahexaenoic acid, EPA-eicosapentaenoic acid, Fish oil, Folic acid, Ginkgo biloba, Ginseng, Iodine, Iron, Magnesium, Manganese, Molybdenum, Niacin, Omega-3, Pantothenic acid, Riboflavin, Saw palmetto, Selenium, Thiamin, Vitamin A, Vitamin B 12, Vitamin B6, Vitamin C, Vitamin D-cholecalciferol, Vitamin E, Vitamin K, and Zinc.

The nutritional analysis of this composition is measured and the results are listed (Referring to FIG. 66). The oral administration of said composition is at a dosage about 3749.465 mg of one daily intake.

### Example V

One female subject of 28 years used the said oral administration with correct dosage every day for one month. During this period, the subject has lost 18 pounds with No Exercise. Most areas of her body have slimmed from the middle torso to the buttocks and hips.

The composition of this one oral capsule is a mixture of a substance that burns calories and fat—energy metabolism. The analysis of this composition is measured and the results is an said exercise metabolism composition wherein said oral dosage is in the form of an internally ingested tablet, a capsule, drops or a suspension and herein an oral administration of said composition is at a dosage of 1 mg to about 4000 mg daily intake.

### REFERENCES

1. Nelson, David L.; Michael M. Cox (2000 Feb. 15). Lehninger Principles of Biochemistry, Third Edition (3 Har/Com ed.). W. H. Freeman. pp. 1200. ISBN 1572599316.
2. a b Lippard, Stephen J.; Jeremy M. Berg (1994). Principles of Bioinorganic Chemistry. Mill Valley, Calif.: University Science Books. pp. 411. ISBN 0935702725.
3. “Dietary mineral”. en.wikipedia.org/wiki/Dietary_mineral. 2005. Retrieved 2011 Jul. 2.
4. ̂ Merck Index, 11th Edition, 1244.
5. ̂ Supplementinfo.org
6. ̂ a b Otten, J J, Hellwig, J P and Meyers, L D., ed (2006). Dietary Reference Intakes: The Essential Guide to Nutrient Requirements. The National Academies Press. ISBN 0-309-10091-7.
7. ̂ Zempleni J, Wijeratne S S, Hassan Y I. (2009). “Biotin”. Biofactors 35 (1): 36-46. doi:10.1002/biof.8. PMID 19319844.
8. ̂ Zempleni J, Mock D M. (1999). “Biotin biochemistry and human requirements.”. J Nutr Biochem. 10 (3): 128-138. doi:10.1016/S0955-2863(98)00095-3. PMID 15539280.
9. ̂ “Biotin”. DSM Nutritional Products. 2009 Aug. 31. Retrieved 2010 Feb. 19. [dead link]
10. ̂ “Biotin”. en.wikipedia.org/wiki/biotin. 2005. Retrieved 2011 Jul. 2.
11. ̂ “Calcium Carbonate”. Medline Plus. National Institutes of Health. 2005 Oct. 1. Archived from the original on 2007 Oct. 17. Retrieved 2007 Dec. 30.
12. ̂ Herbert A. Lieberman, Leon Lachman, Joseph B. Schwartz (1990). Pharmaceutical Dosage Forms: Tablets. New York: Dekker. p. 153. ISBN 0824780442.
13. ̂ “Calcium carbonate”. en.wikipedia.org/wiki/calcium carbonate. 2005. Retrieved 2011 Feb. 2.
14. ̂ “Calcium supplements”. fitnessforworld.com/supplement/calcium.htm. 2011. Retrieved 2011 Jul. 2.
15. ̂ “Nomenclature of Vitamin D. Recommendations 1981. IUPAC-IUB Joint Commission on Biochemical Nomenclature (JCBN)” reproduced at the Queen Mary, University of London website. Retrieved 21 Mar. 2010.
16. ̂ “cholecalciferol” at Dorland's Medical Dictionary
17. ̂ “Cholecalciferol”. en.wikipedia.org/wiki/cholecalciferol. 2007. Retrieved 2011 Jul. 2.
18. ̂ a b Vieth R (May 1999). “Vitamin D supplementation, 25-hydroxyvitamin D concentrations, and safety”. Am. J. Clin. Nutr. 69 (5): 842-56. PMD 10232622.
19. “Chemical Structure of Vitamins and Minerals”.
   a. scientificpsychic.com/health/vitamins. 2011. Retrieved 2011 Jul. 2.
20. “Vitamin D (Calciferol) Description and Overview”.
   a. vitamin-mineral-info.com/vitamin-d-calciferol.php. 2011. Retrieved 2011 Jul. 2.
21. Mertz W. Chromium occurrence and function in biological systems. Physiol Rev 1969; 49:163-239.
22. Mertz W. Chromium in human nutrition: a review. J Nutr 1993; 123:626-33.
23. Mertz W. Interaction of chromium with insulin: a progress report. Nutr Rev 1998; 56:174-7.
24. Porte Jr. D, Sherwin R S, Baron A (editors). Ellengerg & Rifkin's Diabetes Mellitus, 6th Edition. McGraw-Hill, New York, 2003.
25. Schwarz K, Mertz W. Chromium(III) and the glucose tolerance factor. Arch Biochem Biophys 1959; 85:292-5.
26. ̂ Review of Chromium EXPERT GROUP ON VITAMINS AND MINERALS REVIEW OF CHROMIUM, 12 Aug. 2002
27. ̂ “Linus Pauling Institute at Oregon State University”. Retrieved 2008 Nov. 29.
28. ̂ Eastmond D A, Macgregor J T, Slesinski R S (2008). “Trivalent chromium: assessing the genotoxic risk of an essential trace element and widely used human and animal nutritional supplement”. Crit. Rev. Toxicol. 38 (3): 173-90. doi:10.1080/10408440701845401. PMID 18324515.
29. ̂ Stearns D M (2000). “Is chromium a trace essential metal?”. Biofactors 11 (3): 149-62. doi:10.1002/biof.5520110301. PMID 10875302.
30. ̂ Anton S D, Morrison C D, Cefalu W T, et al. (October 2008). “Effects of chromium picolinate on food intake and satiety”. Diabetes Technol. Ther. 10 (5): 405-12. doi:10.1089/dia.2007.0292. PMC 2753428. PMID 18715218.
31. Anderson R A, Bryden N A, Polansky M M. Dietary chromium intake: freely chosen diets, institutional diets and individual foods. Biol Trace Elem Res 1992; 32:117-21.
32. Kozlovsky A S, Moser P B, Reiser S, Anderson R A. Effects of diets high in simple sugars on urinary chromium losses. Metabolism 1986; 35:515-8.
33. Offenbacher E. Promotion of chromium absorption by ascorbic acid. Trace Elem Elect 1994; 11:178-81.
34. Lim T H, Sargent T 3rd, Kusubov N. Kinetics of trace element chromium(III) in the human body. Am J Physiol 1983; 244:R445-54.
35. Lukaski H C. Chromium as a supplement. Annu Rev Nutr 1999; 19:279-302.
36. Davies S, Howard J M, Hunnisett A, Howard M. Age-related decreases in chromium levels in 51,665 hair, sweat, and serum samples from 40,872 patients—implications for the prevention of cardiovascular disease and type II diabetes mellitus. Metabolism 1997; 46:469-73.
37. Institute of Medicine, Food and Nutrition Board. Dietary Reference Intakes for Vitamin A, Vitamin K, Arsenic, Boron, Chromium, Copper, Iodine, Iron, Manganese, Molybdenum, Nickel, Silicon, Vanadium, and Zinc. National Academy Press, Washington, D.C., 2001.
38. Gibson R S. Principles of Nutritional Assessment, 2nd Edition. Oxford University Press, New York, 2005.
39. “Dietary Supplement Fact Sheet—Chromium” ods.od.nih.gov/factsheets/chromium/2011. Retrieved 2011 Jul. 2.
40. “Human Hair Anatomical Chart”. anatomical.com/product.asp_Q_pn_E_{—}9781587791642. 2011. Retrieved 2011 Jul. 2.
41. “The Norwood Scale”. americanhairloss.org/men_hair_loss/the_norwood_scale.asp. 2011. Retrieved 2011 Jul. 2.
42. “Chromium picolinate.png”. en.wikipedia.org/wiki/File:Chromium_picolinate.png. 2005. Retrieved 2011 Jul. 2.
43. ̂“Copper gluconate”. en.wikipedia.org/wiki/Copper_gluconate. 2005. Retrieved 2011 Jul. 2.
44. “Copper gluconate”. vitamins-nutrition.org/vitamins/copper.html. 2011. Retrieved
   a. 2011 Jul. 3.
45. “Copper gluconate”. nutritional-supplement-bible.com/copper-gluconate.html. 2011. Retrieved 2011 Jul. 3.
46. ̂ “Docosahexaenoic acid”. en.wikipedia.org/wiki/Docosahexaenoic_acid. 2006. Retrieved 2011 Jul. 3.
47. Vedin I, et al. (1 Jun. 2008). “Effects of docosahexaenoic acid-rich n-3 fatty acid supplementation on cytokine release”. Am J Clin Nutr 87 (6): 1616-1622. PMID 18541548.
48. Karin Yurko-Mauroa, Deanna McCarthya, Dror Romb, Edward B. Nelsona, Alan S. Ryana, Andrew Blackwellc, Norman Salem Jr.a, Mary Stedman (3 May 2010). “Beneficial effects of docosahexaenoic acid on cognition in age-related cognitive decline”.
49. Meharban Singh (March 2005). “Essential Fatty Acids, DHA and the Human Brain from the Indian Journal of Pediatrics, Volume 72” (PDF). Retrieved Oct. 8, 2007.
50. “Eicosapentaenoic acid”. en.wikipedia.org/wiki/Eicosapentaenoic_acid. 2007. Retrieved 2011 Jul. 3.
51. ̂ a b Yvonne Bishop-Weston. “Plant based sources of vegan & vegetarian Docosahexaenoic acid—DHA and Eicosapentaenoic acid EPA & Essential Fats”. Retrieved 2008 Aug. 5.
52. ̂ NIH Medline Plus. “MedlinePlus Herbs and Supplements: Omega-3 fatty acids, fish oil, alpha-linolenic acid”. Retrieved Feb. 14, 2006.
53. ̂ Peet M, Brind J, Ramchand C N, Shah S, Vankar G K (2001). “Two double-blind placebo-controlled pilot studies of eicosapentaenoic acid in the treatment of schizophrenia”. Schizophrenia Research 49 (3): 243-51. doi:10.1016/S0920-9964(00)00083-9. PMID 11356585.
54. Song C, Zhao S (October 2007). “Omega-3 fatty acid eicosapentaenoic acid. A new treatment for psychiatric and neurodegenerative diseases: a review of clinical investigations”. Expert Opin Investig Drugs 16 (10): 1627-38. doi:10.1517/13543784.16.10.1627. PMID 17922626.
55. Chiu L C; Wan J M. Induction of apoptosis in HL-60 cells by eicosapentaenoic acid (EPA) is associated with downregulation of bcl-2 expression. Cancer Lett 1999 Oct. 18; 145(1-2):17-27
56. ̂ Morris, Martha C.; Sacks, Frank; Rosner, Bernard (1993). “Does fish oil lower blood pressure? A meta-analysis of controlled trials”. Circulation 88 (2): 523-533. PMID 8339414.
57. ̂ Mori, Trevor A.; Bao, Danny Q.; Burke, Valerie; Puddey, Ian B.; Beilin, Lawrence J. (1993). “Docosahexaenoic acid but not eicosapentaenoic acid lowers ambulatory blood pressure and heart rate in humans”. Hypertension 34 (2): 253-260. PMID 10454450.
58. ̂ Augustsson, Katarina; et al. (2003). “A prospective study of intake of fish and marine fatty acids and prostate cancer”. Cancer Epidemiology, Biomarkers & Prevention 12 (1): 64-67. PMID 12540506.
59. ̂ De Deckere, E. A. (1999). “Possible beneficial effect of fish and fish n-3 polyunsaturated fatty acids in breast and colorectal cancer”. Eur J Cancer Prev 8 (3): 213-221. doi: 10.1097/00008469-199906000-00009. PMID 10443950.
60. ̂ Caygill, C. P.; Hill, M. J. (1995). “Fish, n-3 fatty acids and human colorectal and breast cancer mortality”. Eur J Cancer Prev 4 (4): 329-332. doi:10.1097/00008469-199508000-00008. PMID 7549825.
61. “Omega-3 fatty acid”. en.wikipedia.org/wiki/Omega-3_fatty_acid 2004. Retrieved 2011 Jul. 3.
62. ̂ Moghadasian M H (May 2008). “Advances in dietary enrichment with n-3 fatty acids”. Critical Reviews in Food Science and Nutrition 48 (5): 402-10. doi: 10.1080/10408390701424303. PMID 18464030
63. ̂ Leslie G Cleland, Michael J James, and Susanna M Proudman (1992). “Fish oil: what the prescriber needs to know”. Arthritis Research & Therapy 122 (10): 679-81. doi:10.1186/ar1876. PMC 1526555. PMID 16542466.
64. Dyerberg J, Bang H O, Hjorne N (1975). “Fatty acid composition of the plasma lipids in Greenland Eskimos”. Am J Clin Nutr 28 (9): 958-66. PMID 1163480.
65. ̂ Augustsson, Katarina; et al. (2003). “A prospective study of intake of fish and marine fatty acids and prostate cancer”. Cancer Epidemiology, Biomarkers & Prevention 12 (1): 64-67. PMID 12540506.
66. ̂ De Deckere, E. A. (1999). “Possible beneficial effect of fish and fish n-3 polyunsaturated fatty acids in breast and colorectal cancer”. Eur J Cancer Prev 8 (3): 213-221. doi:10.1097/00008469-199906000-00009. PMID 10443950.
67. ̂ Caygill, C. P.; Hill, M. J. (1995). “Fish, n-3 fatty acids and human colorectal and breast cancer mortality”. Eur J Cancer Prev 4 (4): 329-332. doi:10.1097/00008469-199508000-00008. PMID 7549825.
68. ̂ Su, Kuan-Pin; Huang, Shih-Yi; Chiub, Chih-Chiang; Shenc, Winston W. (2003). “Omega-3 fatty acids in major depressive disorder: A preliminary double-blind, placebo-controlled trial”. Eur Neuropsychopharmacol 13 (4): 267-271. doi:10.1016/S0924-977X(03)00032-4. PMID 12888186.
69. ̂ Naliwaiko, K.; Araújo, R. L.; da Fonseca, R. V.; Castilho, J. C.; Andreatini, R.; Bellissimo, M. I.; Oliveira, B. H.; Martins, E. F.; Curi, R.; Fernandes, L. C.; Ferraz, A. C. (April 2004). “Effects of fish oil on the central nervous system: a new potential antidepressant?”. Nutritional Neuroscience (Maney) 7 (2): 91-99. doi:10.1080/10284150410001704525. PMID 15279495.
70. ̂ Green, Pnina; Hermesh, Haggai; Monselisec, Assaf; Maromb, Sofi; Presburgerb, Gadi; Weizman, Abraham (2006). “Red cell membrane omega-3 fatty acids are decreased in nondepressed patients with social anxiety disorder”. Eur Neuropsychopharmacol 16 (2): 107-113. doi:10.1016/j.euroneuro.2005.07.005. PMID 16243493.
71. ̂ Yehuda S., Rabinovitz S., Mostofsky D. I. (2005). “Mixture of essential fatty acids lowers test anxiety”. Nutritional Neuroscience 8 (4): 265-267. doi:10.1080/10284150500445795. PMID 16491653.
72. A Nemets, Boris; Stahl, Ziva; Belmaker, R. H. (2002). “Addition of omega-3 fatty acid to maintenance medication treatment for recurrent unipolar depressive disorder”. Am J Psychiatry 159 (3): 477-479. doi:10.1176/appi.ajp.159.3.477. PMID 11870016.
73. ̂ WebMD: Depression and Fish Oil.
74. “What are the benefits of ginkgo biloba”. livestrong.com/article/368294-what-are-the-benefits-of-ginkgo-biloba. 2011. Retrieved 2011 Jul. 3.
75. ̂ a b Weinmann, S; Roll, S; Schwarzbach, C; Vauth, C; Willich, S N (2010). “Effects of Ginkgo biloba in dementia: systematic review and meta-analysis”. BMC geriatrics 10: 14. doi:10.1186/1471-2318-10-14. PMC 2846949. PMID 20236541
76. ̂ Dekosky, S. T.; Williamson, J. D.; Fitzpatrick, A. L.; Kronmal, R. A.; Ives, D. G.; Saxton, J. A.; Lopez, O. L.; Burke, G. et al. (2008). “Ginkgo biloba for Prevention of Dementia: A Randomized Controlled Trial”. JAMA: the Journal of the American Medical Association 300 (19): 2253-62. doi:10.1001/jama.2008.683. PMC 2823569. PMID 19017911.
77. ̂ a b Snitz, B. E.; O'Meara, E. S.; Carlson, M. C.; Arnold, A. M.; Ives, D. G.; Rapp, S. R.; Saxton, J.; Lopez, O. L. et al. (2009). “Ginkgo biloba for Preventing Cognitive Decline in Older Adults: A Randomized Trial”. JAMA: the Journal of the American Medical Association 302 (24): 2663-70. doi:10.1001/jama.2009.1913
78. ̂ Mahadevan, S.; Park, Y. (2007). “Multifaceted Therapeutic Benefits of Ginkgo biloba L.: Chemistry, Efficacy, Safety, and Uses”. Journal of Food Science 73 (1): R14-9. doi:10.1111/j.1750-3841.2007.00597.x. PMID 18211362.
79. “Ginkgo biloba benefits”. herbwisdom.com/herb-ginkgo-biloba.html. 2011. Retrieved
   a. 2011 Jul. 3.
80. “Ginkgo biloba”. chemspider.com/Search.aspx?q=ginkgo+biloba. 2011. Retrieved
   a. 2011 Jul. 5.
81. “About Ginseng”. asiachi.com/aboutginseng.html. 2011. Retrieved 2011 Jul. 3.
82. “Ginsenosides”. extrasynthese.com/ginsenosides.html. 2011. Retrieved 2011 Jul. 3.
83. “Ginseng Benefits”. herbwisdom.com/herb-ginseng-asian.html. 2011. Retrieved 2011 Jul. 3.
84. “Iodine”. en.wikipedia.org/wiki/Iodine. 2011. Retrieved 2011 Jul. 5.
85. Brown-Grant, K. (1961). “Extrathyroidal iodide concentrating mechanisms”. Physiol Rev. 41: 189.
86. ̂ Spitzweg, C., Joba, W., Eisenmenger, W. and Heufelder, A. E. (1998). “Analysis of human sodium iodide symporter gene expression in extrathyroidal tissues and cloning of its complementary deoxyribonucleic acid from salivary gland, mammary gland, and gastric mucosa”. J Clin Endocrinol Metab. 83 (5): 1746. doi:10.1210/jc.83.5.1746. PMID 9589686.
87. ̂ Banerjee, R. K., Bose, A. K., Chakraborty, T. K., de, S. K. and Datta, A. G. (1985). “Peroxidase catalysed iodotyrosine formation in dispersed cells of mouse extrathyroidal tissues”. J Endocrinol. 2 (2): 159. PMID 2991413
88. “Iodine”. healthsupplementsnutritionalguide.com/Iodine.html. 2011. Retrieved 2011 Jul. 5.
89. “Iron II sulfate”. en.wikipedia.org/wiki/Iron(II)_sulfate. 2006. Retrieved 2011 Jul. 5.
90. “Benefits of Iron Supplement”. ehow.com/about_{—}5070682_benefits-iron-supplement.html. 2011. Retrieved 2011 Jul. 5.
91. “Iron deficiency anemia”. en.wikipedia.org/wiki/Iron_deficiency_anemia. 2006. Retrieved 2011 Jul. 5.
92. “Hair Loss? It May Be Iron Deficiency”. webmd.com/skin-problems-and-treatments/hair-loss/news/20060516/hair-loss-may-be-iron-deficiency. 2011. Retrieved 2011 Jul. 5.
93. “Magnesium: The missing health supplement”. nutritionalmagnesium.org/articles/aging/8-deficiency.html. 2011. Retrieved 2011 Jul. 5.
94. “Magnesium oxide”. en.wikipedia.org/wiki/Magnesium_oxide. 2006. Retrieved 2011 Jul. 5.
95. “Manganese supplements”. vitamins-supplements.org/dietary-minerals/manganese.php. 2011. Retrieved 2011 Jul. 5.
96. “Superoxide Dismutase”. en.wikipedia.org/wiki/Superoxide_dismutase. 2006. Retrieved 2011 Jul. 5.
97. ̂ a b PDB 1VAR; Borgstahl G E, Parge H E, Hickey M J, Johnson M J, Boissinot M, Hallewell R A, Lepock J R, Cabelli D E, Tainer J A (April 1996). “Human mitochondrial manganese superoxide dismutase polymorphic variant Ile58Thr reduces activity by destabilizing the tetrameric interface”. Biochemistry 35 (14): 4287-97. doi:10.1021/bi951892w. PMID 8605177.
98. Schwarz G (December 2005). “Molybdenum cofactor biosynthesis and deficiency”. Cell. Mol. Life Sci. 62 (23): 2792-810. doi:10.1007/s00018-005-5269-y. PMID 16261263.
99. ̂ Smolinsky B, Eichler S A, Buchmeier S, Meier J C, Schwarz G (June 2008). “Splice-specific functions of gephyrin in molybdenum cofactor biosynthesis”. J. Biol. Chem. 283 (25): 17370-9. doi:10.1074/jbc.M800985200. PMID 18411266.
100. “Molybdenum cofactor”. en.wikipedia.org/wiki/Molybdenum_cofactor. 2009. Retrieved 2011 Jul. 5.
101. “Molybdenum”. diet.com/g/molybdenum. 2011. Retrieved 2011 Jul. 5.
102. “Molybdenum”. vitamins-supplements.org/dietary-minerals/molybdenum.php. 2011. Retrieved 2011 Jul. 5.
103. ̂ Richards, Oscar W (1938) ‘The Stimulation of Yeast Proliferation By Pantothenic Acid’ available at <http://www.jbc.org/content/113/2/531.full.pdf>
104. “Pantothenic acid (Oral Route)”. mayoclinic.com/health/drug-information/DR601077. 2011. Retrieved 2011 Jul. 6.
105. “Pantothenic acid”. en.wikipedia.org/wiki/Pantothenic_acid. 2009. Retrieved 2011 Jul. 6.
106. “Pantothenic acid”. vitamin-basics.com/index.php?id=50. 2011. Retrieved 2011 Jul. 6.
107. ̂ Gropper, S. S, Smith, J. L., Groff, J. L. (2009). Advanced nutrition and human metabolism. Belmont, Calif.: Wadsworth, Cengage learning.
108. ̂ a b Jane Higdon, “Pantothenic Acid”, Micronutrient Information Center, Linus Pauling Institute.
109. ̂ a b c d e f g Trumbo, P. R. (2006). Pantothenic Acid. In Shils, M. E., Shike, M., Ross, A. C., Caballero, B., Cousins, R. J. (Eds) Modern Nutrition in Health and Disease. 10th ed. (pp. 462-467) Philadelphia, Pa.: Lippincott Williams & Wilkins.
110. ̂ a b c d e f g h i Gropper, S. S, Smith, J. L., Groff, J. L. (2009). Advanced nutrition and human metabolism. Belmont, Calif.: Wadsworth, Cengage learning.
111. ̂ Linus Pauling Institute at Oregon State University. Micronutrient Information Center. “Pantothenic Acid.” Last accessed 7 Nov. 2010.
112. “Saw palmetto extract”. en.wikipedia.org/wiki/Saw_palmetto_extract. 2009. Retrieved 2011 Jul. 7.
113. “Saw palmetto”. shining-asia.com.tw/paper/plant138-12.html. 2011. Retrieved 2011 Jul. 7.
114. “Saw palmetto fruit extract”. foodchem.com/Herbal_Extracts/Saw_palmetto_Fruit_Extract 2011. Retrieved 2011 Jul. 7.
115. ̂ a b Wadsworth T L, Worstell T R, Greenberg N M, Roselli C E (May 2007). “Effects of dietary saw palmetto on the prostate of transgenic adenocarcinoma of the mouse prostate model (TRAMP)”. The Prostate 67 (6): 661-73. doi:10.1002/pros.20552. PMID 17342743.
116. ̂ Scaglione F, Lucini V, Pannacci M, Caronno A, Leone C (2008). “Comparison of the potency of different brands of Serenoa repens extract on 5alpha-reductase types I and II in prostatic co-cultured epithelial and fibroblast cells”. Pharmacology 82 (4): 270-5. doi:10.1159/000161128. PMID 18849646.
117. ̂ Abe M, Ito Y, Oyunzul L, Oki-Fujino T, Yamada S (April 2009). “Pharmacologically relevant receptor binding characteristics and 5alpha-reductase inhibitory activity of free Fatty acids contained in saw palmetto extract” ([dead link]). Biological & Pharmaceutical Bulletin 32 (4): 646-50. doi:10.1248/bpb.32.646. PMID 19336899.
118. ̂ Habib F K, Ross M, Ho C K, Lyons V, Chapman K (March 2005). “Serenoa repens (Permixon) inhibits the 5alpha-reductase activity of human prostate cancer cell lines without interfering with PSA expression”. International Journal of Cancer 114 (2): 190-4. doi:10.1002/ijc.20701. PMID 15543614.
119. ̂ Wilt T, Ishani A, Mac Donald R., “Serenoa repens for benign prostatic hyperplasia. Cochrane Database Syst Rev. 2002; (3):CD001423. http://www.ncbi.nlm.nih.gov/pubmed/12137626.
120. “Saw palmetto herbal remedies”. health.howstuffworks.com/wellness/natural-medicine/herbal-remedies/saw-palmetto-herbal-remedies.htm2011. Retrieved 2011 Jul. 7.
121. “beta-Sitosterol”. en.wikipedia.org/wiki/Beta-Sitosterol. 2009. Retrieved 2011 Jul. 7.
122. “Selenium”. en.wikipedia.org/wiki/Selenium. 2003. Retrieved 2011 Jul. 7.
123. “What is Selenium Yeast”. livestrong.com/article/229192-what-is-selenium-yeast/. 2011. Retrieved 2011 Jul. 7.
124. “Selenium”. vitamins-supplements.org/dietary-minerals/selenium.php. 2011. Retrieved 2011 Jul. 7.
125. ̂ “Selenium”. Linus Pauling Institute at Oregon State University. Retrieved 2009 Jan. 5.
126. “Vitamin A”. en.wikipedia.org/wiki/Vitamin_A. 2007. Retrieved 2011 Jul. 8.
127. “Vitamin A”. vitamin-basics.com/index.php?id=19. 2011. Retrieved 2011 Jul. 8.
128. “Vitamin A”. vitamins-supplements.org/vitamin-A.php. 2011. Retrieved 2011 Jul. 7.
129. “Thiamine”. en.wikipedia.org/wiki/Thiamine. 2005. Retrieved 2011 Jul. 8.
130. ̂ a b c d e f g h Mahan L K, Escott-Stump S, editors. Krause's food, nutrition, & diet therapy. 10th ed. Philadelphia: W.B. Saunders Company; 2000
131. “Vitamin B1-Thiamine”. vitamin-mineral-info.com/vitamin-b1-thiamin.php. 2011. Retrieved 2011 Jul. 8.
132. “Vitamin B1”. vitamin-basics.com/index.php?id=45. 2011. Retrieved 2011 Jul. 8.
133. “Riboflavin”. en.wikipedia.org/wiki/Riboflavin. 2005. Retrieved 2011 Jul. 8.
134. “Vitamin B2”. vitamin-basics.com/index.php?id=46. 2011. Retrieved 2011 Jul. 8.
135. “Vitamin B2-(Riboflavin)”. vitamins-supplements.org/vitamin-B2-riboflavin.php. 2011. Retrieved 2011 Jul. 7.
136. “Niacin”. en.wikipedia.org/wiki/Niacin. 2005. Retrieved 2011 Jul. 8.
137. “Vitamin B3 (niacin, nicotinic acid, nicotinamide, niacinamide)”. vitamins-supplements.org/vitamin-B3-niacin.php. 2011. Retrieved 2011 Jul. 8.
138. “Niacin: Nicotinic Acid and Nicotinamide”. vitamin-basics.com/index.php?id=49. 2011. Retrieved 2011 Jul. 8.
139. “Pyridoxine”. en.wikipedia.org/wiki/Pyridoxine. 2007. Retrieved 2011 Jul. 8.
140. “Vitamin B6”. vitamin-basics.com/index.php?id=47. 2011. Retrieved 2011 Jul. 8.
141. “Vitamin B6 (pyridoxine)”. vitamins-supplements.org/vitamin-B2-riboflavin.php. 2011. Retrieved 2011 Jul. 8.
142. “Vitamin B12”. en.wikipedia.org/wiki/Vitamin_B12. 2005. Retrieved 2011 Jul. 8.
143. “Vitamin B12 (cyanocobalamin, cobalamin)”. vitamins-supplements.org/vitamin-B12-cyanocobalamin.php. 2011. Retrieved 2011 Jul. 8.
144. “Vitamin B12”. vitamin-basics.com/index.php?id=48. 2011. Retrieved 2011 Jul. 8.
145. “Vitamin C”. en.wikipedia.org/wiki/Vitamin_C. 2003. Retrieved 2011 Jul. 8.
146. ̂ Padayatty, Sebastian J.; Katz, Arie; Wang, Yaohui; Eck, Peter; Kwon, Oran; Lee, Je-Hyuk; Chen, Shenglin; Corpe, Christopher et al. (2003). “Vitamin C as an antioxidant: evaluation of its role in disease prevention.”. Journal of the American College of Nutrition 22 (1): 18-35. PMID 12569111.
147. ̂ a b c d “Vitamin C—Risk Assessment” (PDF). UK Food Standards Agency.
   a. Retrieved 2007 Feb. 19.
148. “Vitamin C”. vitamin-basics.com/index.php?id=43. 2011. Retrieved 2011 Jul. 8.
149. “Vitamin C (ascorbic acid) functions, uses, and health benefits”. vitamins-supplements.org/vitamin-C.php. 2011. Retrieved 2011 Jul. 8.
150. “Tocopherol”. en.wikipedia.org/wiki/Tocopherol. 2005. Retrieved 2011 Jul. 8.
151. “Vitamin E”. vitamin-basics.com/index.php?id=39. 2011. Retrieved 2011 Jul. 8.
152. “Vitamin E (tocopherol) functions, uses, and health benefits”. vitamins-supplements.org/vitamin-E.php. 2011. Retrieved 2011 Jul. 8.
153. “Phylloquinone”. en.wikipedia.org/wiki/Phylloquinone. 2005. Retrieved 2011 Jul. 9.
154. “Vitamin K”. vitamin-basics.com/index.php?id=41. 2011. Retrieved 2011 Jul. 9.
155. ̂ a b Dam, H. (1935). “The Antihæmorrhagic Vitamin of the Chick.: Occurrence And Chemical Nature”. Nature 135 (3417): 652-653. doi:10.1038/135652b0.
156. ̂ Furie B, Bouchard B A, Furie B C (15 Mar. 1999). “Vitamin K-dependent biosynthesis of gamma-carboxyglutamic acid”. Blood 93 (6): 1798-808. PMID 10068650.
157. “Vitamin K”. vitamins-supplements.org/vitamin-K.php. 2011. Retrieved 2011 Jul. 9.
158. “Zinc gluconate”. en.wikipedia.org/wiki/Zincgluconate. 2007. Retrieved 2011 Jul. 9.
159. “Zinc supplements”. vitamins-supplements.org/dietary-minerals/zinc.php. 2011. Retrieved 2011 Jul. 9.
160. “Vitamin B9”. en.wikipedia.org/wiki/Vitamin_B9. 2004. Retrieved 2011 Sep. 30.
161. ̂ Ural, Serdar H. (2008 November). “Folic Acid and Pregnancy.”. Kid's Health.
162. “Vitamin B9”. vitamin-basics.com/index.php?id=51. 2011. Retrieved 2011 Sep. 30.
163. ̂ a b c d Weinstein, S J et al. (2003). “Null Association Between Prostate Cancer and Serum Folate, Vitamin B6, Vitamin B12, and Homocysteine” (PDF). Cancer Epidemiology, Biomarkers, & Prevention 12 (11): 1271-1272. Retrieved 1 Dec. 2010.
164. ̂ “Dietary Supplement Fact Sheet: Folate.”. Office of Dietary Supplements, National Institutes of Health.
165. ̂ Williams, J D; Jacobson, M K (2010). “Photobiological implications of folate depletion and repletion in cultured human keratinocytes.”. Journal of Photochemistry and Photobiology B: Biology 99 (1): 49-61. doi:10.1016/j.jphotobiol.2010.02.003. PMC 2862485. PMID 20211567.

What is claimed is:

1. Promoting hair growth and preventing hair loss thereof administering a composition of mixed vitamin and herbal supplements wherein said concentration of a mixed vitamin and herbal supplements is about 0.1%.

2. The composition of claim 1, wherein said composition of vitamin and herbal supplements are a mixture of Biotin, Calcium, Chromium, Copper, DHA-docosahexaenoic acid, EPA-eicosapentaenoic acid, Fish oil, Folic acid, Ginkgo biloba, Ginseng, Iodine, Iron, Magnesium, Manganese, Molybdenum, Niacin, Omega-3, Pantothenic acid, Riboflavin, Saw palmetto, Selenium, Thiamin, Vitamin A, Vitamin B12, Vitamin B6, Vitamin C, Vitamin D-cholecalciferol, Vitamin E, Vitamin K, and Zinc.

3. The composition of claim 2, wherein said composition comprises a suitable carrier, solvent and/or emulgent.

4. The composition of claim 2, wherein said composition is administered in the form of an oral dosage.

5. The composition of claim 2, wherein said oral dosage is in the form of an internally ingested tablet, a capsule, drops or a suspension.

6. The composition of claim 1, wherein said oral dosage promotes healthy micronutrient growth and development for deprived areas of the body.

7. The composition of claim 1, wherein an oral administration of said composition is at a dosage of about 37 mg to about 7500 mg per day.

8. A method of promoting hair growth and preventing hair loss consisting of administering in humans and animals wherein the minimum concentration of said mixed vitamin and herbal supplements is about 0.1%. wherein said composition is administered in the form of an oral dosage as in the form of an internally ingested tablet, a capsule, drops or a suspension wherein said oral dosage promotes healthy micronutrient growth and development for deprived areas of the body wherein an oral dosage 1, an oral dosage 2, an oral dosage 3, an oral dosage 4, an oral dosage 5, an oral dosage 6, and an oral dosage 7, are taken daily from 1 day to about 180 days wherein an oral administration of said oral dosage 1, said oral dosage 2, said oral dosage 3, said oral dosage 4, said oral dosage 5, said oral dosage 6, and said oral dosage 7 is at a dosage of about 37 mg to about 7500 mg per day.

9. The method according to claim 1, wherein said oral dosage 1 comprises a composition of vitamin and herbal supplements are a mixture of wherein the amount of said minimum concentration about 0.1%, and wherein said composition is administered in the form of an oral dosage comprising Biotin, Calcium, Chromium, Copper, Folic acid, Iodine, Iron, Magnesium, Manganese, Molybdenum, Niacin, Pantothenic acid, Riboflavin, Selenium, Thiamin, Vitamin A, Vitamin B12, Vitamin B6, Vitamin C, Vitamin D-cholecalciferol, Vitamin E, Vitamin K, and Zinc wherein said minimum concentration is about 3 mg wherein said oral dosage is in the form of an internally ingested tablet, a capsule, drops or a suspension and herein an oral administration of said composition is at a dosage of about 3 mg to about 618 mg per day.

10. The method according to claim 1, wherein said oral dosage 2 comprises a composition of vitamin and herbal supplements are a mixture of wherein the amount of said minimum concentration about 0.1%, and wherein said composition is administered in the form of an oral dosage comprising DHA-docosahexaenoic acid, EPA-eicosapentaenoic acid, Fish oil, and Omega-3 wherein said minimum concentration is about 24 mg wherein said oral dosage is in the form of an internally ingested tablet, a capsule, drops or a suspension and herein an oral administration of said composition is at a dosage of about 24 mg to about 4800 mg per day.

11. The method according to claim 1, wherein said oral dosage 3 comprises a composition of vitamin and herbal supplements are a mixture of wherein the amount of said minimum concentration about 0.1%, and wherein said composition is administered in the form of an oral dosage comprising Calcium and wherein said minimum concentration is about 6 mg wherein said oral dosage is in the form of an internally ingested tablet, a capsule, drops or a suspension and herein an oral administration of said composition is at a dosage of about 6 mg to about 1200 mg per day.

12. The method according to claim 1, wherein said oral dosage 4 comprises a composition of vitamin and herbal supplements are a mixture of wherein the amount of said minimum concentration about 0.1%, and wherein said composition is administered in the form of an oral dosage comprising Vitamin D—cholecalciferol and wherein said minimum concentration is about 0.00125 mg wherein said oral dosage is in the form of an internally ingested tablet, a capsule, drops or a suspension and herein an oral administration of said composition is at a dosage of about 00125 mg to about 0.25 mg per day.

13. The method according to claim 1, wherein said oral dosage 5 comprises a composition of vitamin and herbal supplements are a mixture of wherein the amount of said minimum concentration about 0.1%, and wherein said composition is administered in the form of an oral dosage comprising Saw Palmetto and wherein said minimum concentration is about 1.6 mg wherein said oral dosage is in the form of an internally ingested tablet, a capsule, drops or a suspension and herein an oral administration of said composition is at a dosage of about 1.6 mg to about 320 mg per day.

14. The method according to claim 1, wherein said oral dosage 6 comprises a composition of vitamin and herbal supplements are a mixture of wherein the amount of said minimum concentration about 0.1%, and wherein said composition is administered in the form of an oral dosage comprising Ginseng and wherein said minimum concentration is about 2.5 mg wherein said oral dosage is in the form of an internally ingested tablet, a capsule, drops or a suspension and herein an oral administration of said composition is at a dosage of about 2.5 mg to about 500 mg per day.

15. The method according to claim 1, wherein said oral dosage 7 comprises a composition of vitamin and herbal supplements are a mixture of wherein the amount of said minimum concentration about 0.1%, and wherein said composition is administered in the form of an oral dosage comprising Ginkgo Biloba and wherein said minimum concentration is about 0.6 mg wherein said oral dosage is in the form of an internally ingested tablet, a capsule, drops or a suspension and herein an oral administration of said composition is at a dosage of about 0.6 mg to about 60 mg per day.
